(19) United States
(12) Patent Application Publication
(10) Pub. No.: US 2023/0270668 A1
(43) Pub. Date: Aug. 31, 2023

(54) DRY FORMULATIONS OF ANTI-SARS-COV-2 VIRUS ANTIBODIES AND COMPOSITIONS AND METHODS OF USE THEREOF

(71) Applicants: AUGMENTA BIOWORKS, INC., Menlo Park, CA (US); TFF PHARMACEUTICALS, INC., Fort Worth, TX (US); BOARD OF REGENTS, THE UNIVERSITY OF TEXAS SYSTEM, Austin, TX (US)

(72) Inventors: Christopher J. EMIG, Menlo Park, CA (US); Steven J. HENRY, Menlo Park, CA (US); Adela Anne VITUG, Menlo Park, CA (US); Dale J. CHRISTENSEN, Cary, NC (US); Zhengrong CUI, Austin, TX (US); Haiyue XU, Austin, TX (US); Robert O. WILLIAMS, III, Austin, TX (US)

(21) Appl. No.: 18/045,745

(22) Filed: Oct. 11, 2022

Related U.S. Application Data
(60) Provisional application No. 63/254,487, filed on Oct. 11, 2021.

(51) Int. Cl. A61K 9/00; C07K 16/10; A61P 31/14

(52) U.S. Cl. CPC A61K 9/0075; C07K 16/1018; A61P 31/14

(57) ABSTRACT

The invention generally encompasses a human-derived monoclonal antibody compositions and methods comprising a dry powder formulation for neutralizing SARS-CoV-2 and known variants thereof in a patient, comprising administering to the patient a dry powder formulation comprising the human-derived monoclonal antibody. The invention further encompasses compositions and methods comprising a dry powder formulation for treating or preventing COVID-19 and/or at least one symptom associated with COVID-19 in a patient, comprising administering to the patient a dry powder formulation comprising the human-derived monoclonal antib.

The present application claims the benefit of priority to U.S. Provisional Application No. 63/254,487, filed on Oct. 11, 2021, the entire contents of which are hereby incorporated by reference.

This application contains a Sequence Listing XML, which has been submitted electronically and is hereby incorporated by reference in its entirety. Said XML Sequence Listing, created on Oct. 11, 2022, is named UTSBP1321US.xml and is 10,867 bytes in size.

### FIELD OF THE INVENTION

The invention generally encompasses monoclonal antibody compositions and methods comprising a dry powder formulation for neutralizing SARS-CoV-2 and known variants thereof in a patient, comprising administering to the patient a dry powder formulation comprising the monoclonal antibody. In certain embodiments, the invention further encompasses compositions and methods comprising a dry powder formulation for treating or preventing COVID-19 and/or at least one symptom associated with COVID-19 in a patient, comprising administering to the patient a dry powder formulation comprising a human-derived monoclonal antibody. In other embodiments, the invention encompasses a dry powder formulation including an antibody made using thin film freeze drying that is stable at ambient temperatures that may be reconstituted, for example, for injection.

### BACKGROUND OF THE INVENTION

Infections caused by a novel coronavirus began emerging in late 2019 that became known as the Severe Acute Respiratory Syndrome Coronavirus 2 (SARS-CoV-2) virus. This Coronavirus Disease 2019 (COVID-19) resulted in a global pandemic that has resulted in the loss of millions of lives globally. The SARS-CoV-2 virus is related to other coronaviruses that caused previous epidemics with Severe Acute Respiratory Syndrome (SARS-CoV) in 2002-2003 and the Middle East Respiratory Syndrome (MERS) in 2012 (Miller et al., 2020; Wu et al., 2020). The COVID-19 pandemic has resulted in more deaths than other coronavirus epidemics or pandemics (Gurwitz, 2020; Miller et al., 2020; Xue et al., 2020) and has had profound effects on the lives of people around the world.

One key mitigation strategy employs neutralizing monoclonal antibodies (mAbs) to treat or protect against SARS-CoV-2 infection. Routes for discovery of new SARS-CoV-2 neutralizing antibodies include isolation of antibody sequences from patients who have recovered from SARS-CoV-2 or SARS-CoV, inoculation and isolation of humanized mice, or the use of phage or other library display technology. Since the outbreak, multiple mAb products have been granted Emergency Use Authorization, but all are administered via infusion or injection. Several groups have published methods for isolating, sequencing and cloning antibody genes from single B cells from primary patient samples, then expressing antibody protein for characterization.

Since SARS-CoV-2 is primarily a pulmonary disease, early treatment with neutralizing mAb therapy could provide a mechanism to neutralize the virus to treat people who may become exposed to infected patients or to treat patients soon after a positive diagnosis of COVID-19 disease. By initiating treatment early in the disease cycle, it may be possible to alter the course of the disease and reduce the hospitalization rate and mortality that results from the disease. Furthermore, since only a fraction of systemically administered mAbs are transported into the pulmonary compartment where viral particles are released early in the disease, delivery of neutralizing mAbs directly to the lung holds the potential to reduce the dose needed to achieve the same efficacy as systemically delivered mAbs.

The inventors have developed a thin-film freeze-drying (TFFD) process, which is a rapid freezing technology originally studied to enhance the solubility of poorly water soluble compounds, and was recently used to prepare stable submicron protein particles. In the process of TFFD, droplets of drug formulation are rapidly frozen upon impact with a cryogenically-cooled substrate to form thin films in less than a second. These thin films are then lyophilized to remove solvent in the formulation.

The inventors have developed a new inhalable dry powder antibody made using thin-film freezing that can be used to treat, for example, COVID-19 disease.

### SUMMARY OF THE INVENTION

In various embodiments, the invention generally encompasses human-derived monoclonal antibody formulations and compositions and methods comprising a dry powder formulation for neutralizing SARS-CoV-2 and known variants thereof in a patient, comprising administering to the patient a dry powder composition comprising a human-derived monoclonal antibody by oral inhalation. The formulations and compositions may be delivered by oral administration in such a manner to achieve deep lung penetration.

In various embodiments, the invention generally encompasses human-derived monoclonal antibody formulations and compositions and methods comprising a dry powder formulation for neutralizing SARS-CoV-2 and known variants thereof in a patient, comprising intranasally administering to the patient a dry powder composition comprising a human-derived monoclonal antibody.

In another embodiment, the invention encompasses a method for neutralizing SARS-CoV-2 virus in a patient, comprising: (a) obtaining a dry powder composition comprising human-derived monoclonal antibody, AUG-3387; (b) suspending the dry power in pharmaceutically acceptable liquid to form a suspension; and (c) intranasally administering the suspension to the patient.

In another embodiment, the invention encompasses a method for neutralizing SARS-CoV-2 virus in a patient, comprising: (a) obtaining a dry powder composition comprising human-derived monoclonal antibody, AUG-3387; (b) suspending the dry power in pharmaceutically acceptable liquid to form a suspension; and (c) administering the suspension to the patient via oral inhalation.

In another embodiment, the inventions encompass methods of making a powder composition comprising a human-derived monoclonal antibody, AUG-3387, wherein the powder is comprised in a liquid suspension; and administering the suspension to the patient via oral inhalation. In some embodiments, a pharmaceutically acceptable liquid for use according to the embodiments comprises sterile water or saline solution.

In another embodiment, the inventions encompass methods of making a powder composition comprising a human-derived monoclonal antibody, AUG-3387, wherein the powder is comprised in a liquid suspension; and intranasally administering the suspension to the patient. In some embodiments, a pharmaceutically acceptable liquid for use according to the embodiments comprises sterile water or saline solution.

In another embodiment, the invention encompasses a dry powder composition comprising less than 5% water. In some embodiments, the dry powder composition comprises less than 4% water. In some embodiments, the dry powder composition comprises less than 3% water. In some embodiments, the dry powder composition comprises less than 2% water. In some embodiments, the dry powder composition comprises less than 1% water. In some embodiments, the dry powder composition is essentially free of water. In some embodiments, the dry powder composition is prepared from a liquid composition.

In certain embodiments, a dry powder composition of the invention is comprised of particles having an average diameter of about 0.1 to about 100 μm. For example, the powder can have an average diameter of about 0.1 to about 100 μm, about 1 to about 50 μm, about 5 to about 20 μm or about 5 to about 15 μm.

In some embodiments, the dry powder composition further comprises an excipient. In some embodiments, the excipient is a salt. In some embodiments, the excipient is a sugar. In some embodiments the excipient is a buffer. In some embodiments, the excipient is a detergent. In some embodiments the excipient is a polymer. In some embodiments, the excipient is an amino acid. In some embodiments, the excipient is a preservative. In some embodiments, the excipient is disodium edetate, sodium chloride, sodium citrate, sodium succinate, sodium hydroxide, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, tri-n-butyl phosphate, ascorbic acid, cysteine hydrochloride, hydrochloric acid, hydrogen citrate, trisodium citrate, guanidine hydrochloride, mannitol, lactose, sucrose, agarose, sorbitol, maltose, trehalose, surfactants, polysorbate 80, polysorbate 20, poloxamer 188, sorbitan monooleate, TRITON N101, m-cresol, benzyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco's modified eagles medium, hydrocortisone, neomycin, Von Willebrand factor, gluteraldehyde, benzethonium chloride, white petroleum, p-aminophenyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer.

In certain embodiments, a dry powder composition of the invention comprises from about 50% to about 99% (e.g. 60%, 70%, 80%, or 90% to 99%) wt/wt of an excipient. In some other embodiments, the dry powder composition comprises less than 3% wt/wt of an excipient. In some embodiments, the dry powder composition comprises less than 2% wt/wt of an excipient. In some embodiments, the dry powder composition comprises less than 1% wt/wt of an excipient. In some embodiments, the dry powder composition is essentially free of excipients. In some embodiments, the dry powder composition is free of excipients.

In some embodiments, the method for neutralizing SARS-CoV-2 and/or treating COVID-19 infection and/or reducing at least one symptom associated with COVID-19 infection comprises administering an effective amount of a dry powder formulation of a human-derived monoclonal antibody, AUG-3387, to patient in need thereof.

In other embodiments, the invention encompasses a method for inhibiting replication of SARS-CoV-2 in a mammal, for example, a human or non-human primate, comprising administering an effective amount of a human-derived monoclonal antibody, AUG-3387 to mammal in need thereof.

In other embodiments, the invention encompasses a method of treating or preventing a COVID-19 in a patient in need thereof. In some embodiments, the method of treating or preventing COVID-19 may include treating at least one symptom of COVID-19.

In some embodiments, the invention encompasses, pulmonary administration by inhalation of a dry powder formulation comprising a human-derived monoclonal antibody, AUG-3387. The dry powder composition may be administered via oral administration to the lungs or intranasally. In some embodiments, the dry powder composition is administered using an inhaler. In some embodiments, the inhaler comprises a pressurized canister. In some embodiments, the inhaler comprises a pump bottle. In some embodiments, the inhaler comprises a syringe.

In some embodiments, the present disclosure provides a spray comprising a vessel comprising dry powder formulation comprising a human-derived monoclonal antibody, AUG-3387 and an applicator capable of dispersing the dry powder formulation into the nasal or mouth cavity. In some embodiments, the vessel may be configured to delivery to the nasal cavity. In other embodiments, the vessel is designed to delivery to the lungs via the mouth. In some embodiments, the applicator comprises a pressurized canister. In some embodiments, the applicator comprises a pump bottle. In some embodiments, the applicator comprises a syringe.

In some embodiments, the present invention encompasses a kit comprising a dry powder formulation and an applicator capable of dispersing the dry powder formulation into either the mouth or the nasal cavity, wherein the dry powder formulation comprises a human-derived monoclonal antibody, AUG-3387. In some embodiments, the vessel may be configured to delivery to the nasal cavity. In other embodiments, the vessel is designed to delivery to the lungs via the mouth.

Other embodiments, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

In one embodiment, the invention encompasses a dry powder formulation for neutralizing SARS-CoV-2 and known variants thereof in a subject in need thereof comprising administering to the patient the dry powder composition comprising a human-derived monoclonal antibody AUG-3387.

In another embodiment, the invention encompasses a method for neutralizing SARS-CoV-2 virus in a subject in need thereof, comprising administering to said subject a dry powder formulation comprising human-derived monoclonal antibody AUG-3387.

In another embodiment, the invention encompasses a method of treating or preventing COVID-19 disease in a subject in need thereof comprising administering to said subject a dry powder formulation comprising human-derived monoclonal antibody AUG-3387.

In another embodiment, the invention encompasses a method for inhibiting replication of SARS-CoV-2 in a mammal comprising administering to a mammal in need thereof an effective amount of a human-derived monoclonal antibody AUG-3387.

In another embodiment, the invention encompasses a dry powder formulation including an isolated recombinant monoclonal antibody that specifically binds to a Severe Acute Respiratory Syndrome Coronavirus 2 (SARS-CoV-2) virus and/or a SARS-CoV-2 virus envelope glycoprotein, wherein the antibody neutralizes SARS-CoV-2 virus in vitro with an IC₅₀ less than or equal to about 10⁻⁹M and wherein the antibody is of SEQ ID NO: 1.

In another embodiment, the invention encompasses a dry powder formulation including an antibody made using thin film freeze drying process, wherein the dry powder formulation is stable at ambient temperatures (e.g., 20-25° C.).

In certain embodiments, the dry powder formulation may be reconstituted for administration, for example, by injection.

In certain embodiments the dry powder formulation comprises less than 5% water.

In certain embodiments the dry powder formulation comprises less than 2% water.

In certain embodiments the dry powder formulation comprises particles having an average diameter of about 0.1 to about 100 μm.

In certain embodiments the dry powder formulation comprises particles having an average diameter of about 1 to about 50 μm.

In certain embodiments the dry powder formulation comprises particles having an average diameter of about 5 to about 15 μm.

In certain embodiments the dry powder formulation further comprises an excipient.

In certain embodiments the dry powder formulation is suitable for administration by inhalation.

In certain embodiments the inhalation is nasal administration.

In certain embodiments the inhalation is oral administration.

In certain embodiments, the isolated monoclonal antibody has one or more of the following characteristics:
(a) is a fully human monoclonal antibody;
(b) binds to SARS-CoV-2 virus E with a dissociation constant (K_{D}) of less than 10⁻⁷M, as measured in a surface plasmon resonance assay; or
(c) may or may not demonstrate a change in dissociative half-life (t1/2) at pH 5 or pH 6 relative to pH 7.4.

### BRIEF DESCRIPTION OF THE DRAWINGS

FIG. 1 illustrates an exemplary serological profile of seven subjects against six SARS-CoV-2 antigens.

FIG. 2 illustrates illustrative single antibody secreting cells are assayed for their ability to bind various optically encoded antigens in their proximity.

FIGS. 3a-c illustrate (a) Raster of plate at 5× showing antigen specific B cells with signature reaction-diffusion pattern. (b,c) Before and after 20× false color images of automated capture of an S1-RBD specific plasma cell. Blue indicates antigen beads displaying S1 RBD protein, while green indicates beads displaying S2 protein. Magenta is a cell specific stain.

FIG. 4 illustrates exemplary output from a SingleCyte Screen. For each cell, the confidence of an antigen specific interaction is determined by the amount of secondary antibody signal that overlaps an antigen-specific bead image in the proximity of each cell. A score of 0 represents a 50% chance of antigen specificity, with 1.0 representing a 100% certainty.

FIG. 5 illustrates single domain affinity of AUG-3387 binding domain expressed as an ScFv.

FIG. 6 illustrates the susceptibility of AUG-3387 to mutational escape. AUG-3387 was profiled against the S1 and RBD portions of the original Wuhan-1 strain of SARS-CoV-2, RBD's corresponding to WHO designated dominant strains of concern, and S1 mutants known to affect the potency of currently approved therapeutic antibodies. AUG-3387 binds every S1 and RBD of SARS-CoV-2 tested with a binding EC₅₀<200 ng/ml, but only very weakly to SARS-CoV-1 (binding EC₅₀>100 ug/ml, not shown).

FIGS. 7a-b illustrate exemplary neutralization of SARS-CoV-2 by AUG-3387 and AUG-3705.

FIG. 8 illustrates exemplary neutralization of SARS-CoV-2 B.1.617.2 (Delta) pseudovirus by AUG-3387.

FIG. 9 illustrates aerodynamic particle size distribution of AUG-3387 using RS00 high-resistance DPI at a flow rate of 60 L/min (n=3).

FIG. 10 illustrates an exemplary Gel electropherogram from Bio-Rad Experion run on AUG-3387.11 and AUG-3387.13 (native in lanes 1 and 2, reducing in lanes 5 and 6) and AUG-3387 as expressed in Expi-293T (lane 3 and 7) and CHO (lane 4 and 8).

FIG. 10 illustrates exemplary dry powder formulations of AUG-3387 (AUG-3387.11 and AUG-3387.13) bind to SARS-CoV-2 variants at the same concentrations as the PBS formulation.

FIG. 11 illustrates dry powder formulations of AUG-3387 (AUG-3387.11 and AUG-3387.13) bind to SARS-CoV-2 variants at the same concentrations as the PBS formulation indicating that no loss of binding occurred after the TFF processing to create dry powder formulations.

FIG. 12 illustrates AUG-3387 and dry powder formulations AUG-3387.11 and AUG-3387.13 demonstrate neutralization of SARS-CoV-2 Wuhan-1 pseudovirus at the same concentration as the PBS formulation.

FIG. 13 illustrates the viral load in the lungs of hamsters inoculated with SARS-CoV-2 24 hours BEFORE administration of AUG-3387 with either three daily doses of a dry powder (DPI) at 1 or 0.3 mg/kg or a single IP dose of a liquid formulation of antibody at 10 or 3 mg/kg.

FIG. 14 illustrates the AUG-3387/AUG-3705 sequence of the variable heavy chain CDRs and variable light chain CDRs.

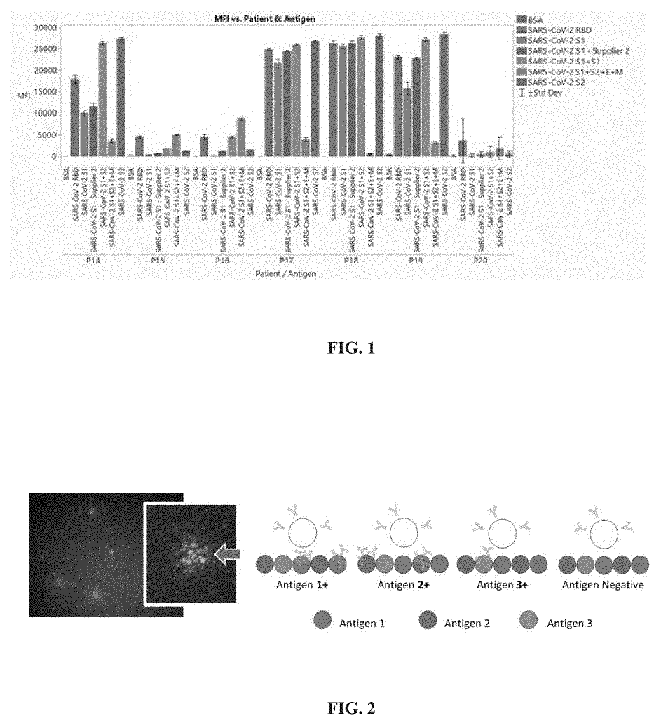

### DETAILED DESCRIPTION OF THE INVENTION

### I. Definitions

The abbreviations used herein have their conventional meaning within the chemical and biological arts.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

The term “adjuvant” is used in accordance with its plain ordinary meaning within immunology and refers to a substance that is commonly used as a component of an immunogenic composition. Adjuvants may increase an antigen specific immune response in a subject when administered to the subject with one or more specific antigens as part of an immunogenic composition. In some embodiments, an adjuvant accelerates an immune response to an antigen. In some embodiments, an adjuvant prolongs an immune response to an antigen. In some embodiments, an adjuvant enhances an immune response to an antigen. In some embodiments, an adjuvant is an aluminum adjuvant.

The term “administer (or administering) a composition” means administering a composition that prevents or treats an infection in a subject. Administration may include, without being limited by mechanism, allowing sufficient time for the immunogenic composition to induce an immune response in the subject or to reduce one or more symptoms of a disease.

The term “amino acid” refers to naturally occurring and synthetic amino acids, as well as amino acid analogs. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. An oligomer comprising amino acid mimetics is a peptidomimetic. A peptidomimetic moiety is a monovalent peptidomimetic.

The term “associated” or “associated with” as used herein to describe a disease (e.g. a virus associated disease) means that the disease is caused by, or a symptom of the disease is caused by, what is described as disease associated or what is described as associated with the disease. As used herein, what is described as being associated with a disease, if a causative agent, could be a target for treatment of the disease.

The terms, “bind,” “bound,” “binding,” and other verb forms thereof are used in accordance with their plain ordinary meaning within Enzymology and Biochemistry and refer to the formation of one or more interactions or contacts between two compositions that may optionally interact. Binding may be intermolecular or intramolecular.

By “co-administer” it is meant that a composition described herein is administered at the same time, just prior to, or just after the administration of one or more additional therapies, for example infection therapies such as antiviral drugs or antibody formulations. The compositions of the embodiments can be administered alone or can be co-administered to the patient. Co-administration is meant to include simultaneous or sequential administration of the compounds individually or in combination (more than one composition). The compositions of the present embodiments can be delivered by transdermally, by a topical route, transcutaneously, formulated as solutions, suspensions, emulsions, gels, creams, ointments, pastes, jellies, paints, powders, and aerosols.

The term “contacting” may include allowing two items to react, interact, or physically touch, wherein the two species may be a composition (e.g. an immunogenic composition) as described herein and a cell, antibody, virus, virus particle, protein, enzyme, or patient. In some embodiments contacting includes allowing a composition described herein to interact with a protein or enzyme that is involved in a signaling pathway. In some embodiments contacting includes allowing a composition described herein to interact with a component of a subject's immune system involved in developing immunity to a component of the composition.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of infection or one or more symptoms of infection in the absence of a composition (e.g. an immunogenic composition) as described herein (including embodiments).

“Disease” or “condition” refer to a state of being or health status of a patient or subject capable of being treated with the compositions or methods provided herein. In some embodiments, the disease is a disease related to (e.g. caused by) an infectious agent (e.g. bacterium or virus).

The terms “dose” and “dosage” are used interchangeably herein. A dose refers to the amount of active ingredient given to an individual at each administration. For the present methods and compositions provided herein, the dose may generally refer to the amount of disease treatment. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

An “effective amount” is an amount sufficient for a composition or formulation (e.g. comprising an antibody) to accomplish a stated purpose relative to the absence of the composition (e.g. achieve the effect for which it is administered, treat a disease (e.g. reverse or prevent or reduce severity), reduce spread of an infectious disease or agent, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.”

As defined herein, the term “inhibition”, “inhibit”, “inhibiting” and the like in reference to a protein-inhibitor or interaction means negatively affecting (e.g. decreasing) the activity or function of the protein. In some embodiments inhibition refers to reduction of a disease or symptoms of disease. In some embodiments inhibition refers to reduction of the growth, proliferation, or spread of an infectious agent (e.g. bacterium or virus). In some embodiments inhibition refers to preventing the infection of a subject by an infectious agent (e.g. bacterium or virus). In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating the signaling pathway or enzymatic activity or the amount of a protein.

As used herein, the term “intranasally administering” means administration such that the majority of the administered composition is deposited in the nasal cavity, and preferably in contact with nasal epithelium. Thus, in some embodiments, intranasal administration is directly applied through the nostrils and results in minimal deposition of administered compositions in the mouth, throat or lungs of a subject. In certain aspects, a composition is selectively deposited in the posterior nasal cavity of a subject.

The term “isolated” refers to a nucleic acid, polynucleotide, polypeptide, protein, or other component that is partially or completely separated from components with which it is normally associated (other proteins, nucleic acids, cells, etc.). In some embodiments, an isolated polypeptide or protein is a recombinant polypeptide or protein.

The term “modulator” refers to a composition that increases or decreases the level of a target (e.g. molecule, cell, bacterium, virus particle, protein) or the function of a target or the physical state of the target.

The term “modulate” is used in accordance with its plain ordinary meaning and refers to the act of changing or varying one or more properties. “Modulation” refers to the process of changing or varying one or more properties. For example, as applied to the effects of a modulator on a target, to modulate means to change by increasing or decreasing a property or function of the target or the amount of the target.

“Patient” or “subject in need thereof” refers to a living organism suffering from or prone to a disease or condition that can be treated by administration of a composition (e.g. pharmaceutical composition or formulation) as provided herein. Non limiting examples include humans, other mammals, bovines, rats, mice, dogs, monkeys, goat, sheep, cows, deer, and other non-mammalian animals. In some embodiments, a patient is human. In some embodiments, a patient or subject in need thereof, refers to a living organism (e.g. human) at risk of developing, contracting, or having a disease or condition associated with an infectious agent (e.g. virus).

“Pharmaceutically acceptable excipient” and “pharmaceutically acceptable carrier” refer to a substance that aids the administration of an active agent to or absorption by a subject and can be included in the compositions of the present embodiments without causing a significant adverse toxicological effect on the patient. Non limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compositions of the embodiments. One of skill in the art will recognize that other pharmaceutical excipients are useful in the embodiments. In embodiments, an excipient is a salt, sugar (saccharide), buffer, detergent, polymer, amino acid, or preservative. In embodiments, the excipient is disodium edetate, sodium chloride, sodium citrate, sodium succinate, sodium hydroxide, Sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, mannitol, lactose, sucrose, agarose, sorbitol, maltose, trehalose, surfactants, polysorbate 80, polysorbate 20, poloxamer 188, sorbitan monooleate, TRITON N101, m-cresol, benzyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco's modified eagles medium, hydrocortisone, neomycin, Von Willebrand factor, gluteraldehyde, benzethonium chloride, white petroleum, p-aminophenyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer.

The term “preparation” is intended to include the formulation of the active compound with encapsulating material as a carrier providing a capsule-like structure in which the active component with or without other carriers, is surrounded by a carrier, which is thus in association with it.

The term “preventing” or “prevention” refers to any indicia of success in protecting a subject or patient (e.g. a subject or patient at risk of developing a disease or condition) from developing, contracting, or having a disease or condition (e.g. an infectious disease or a symptom or disease associated with an infectious agent), including preventing one or more symptoms of a disease or condition or diminishing the occurrence, severity, or duration of any symptoms of a disease or condition following administration of a prophylactic or preventative composition as described herein.

A “reduction” of a symptom or symptoms (and grammatical equivalents of this phrase) means decreasing of the severity or frequency of the symptom(s), or elimination of the symptom(s). A “prophylactically effective amount” of a composition (e.g., an immunogenic composition) is an amount of a composition that, when administered to a subject, will have the intended prophylactic effect, e.g., preventing or delaying the onset (or reoccurrence) of an injury, disease (e.g. infectious disease), pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses (e.g. prime-boost). Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickax, Dosage Calculations (1999); and Remington: The Science and Practice of Pharmacy, 20th Edition, 2003, Gennaro, Ed., Lippincott, Williams & Wilkins).

The terms “treating” or “treatment” refers to any indicia of success in the treatment or amelioration of disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat a disease associated with (e.g. caused by) an infectious agent (e.g. a coronavirus). The term “treating” and conjugations thereof, include prevention of pathology, condition, or disease.

The invention generally encompasses dry powder formulations of AUG-3387.

In certain embodiments, a SingleCyte® system was used to isolate a new mAb, AUG-3387, that displays potent binding to the SARS-CoV-2 S-protein. In certain embodiments, binding to and neutralization of both pseudovirus and Wuhan-1 Coronavirus demonstrated the potential utility of AUG-3387 for treatment of COVID-19 disease. In various embodiments, AUG-3387 demonstrated potent binding to the Alpha, Beta, Gamma, Delta, Lamda and Mu variants suggesting that the antigen site is conserved and has not been mutated in the variants of concern or newly emerging Lamda and Mu variants. In certain embodiments, the neutralization activity validates that the binding is at a site that prevents the virus from binding the hACE2 receptor and cells. In certain embodiments, the retained activity against these variants is in contrast to the reduced susceptibility of Bamlanivimab and Etesevimab, which show greater than 250-fold reduced binding and neutralization activity against the Beta and Gamma variants (https://www.fda.gov/media/145802/download).

In certain embodiments, the mAb, AUG-3387, was formulated as a room temperature stable dry powder utilizing the thin-film freezing process. In certain embodiments, the room temperature stability may allow for distribution to geographic locations where SARS-CoV-2 continues to spread but that do not have the capability of distributing injectable formulations that require cold chain distribution and storage. In certain embodiments, the formulations prepared using the TFF process retain full binding activity of the input mAb solutions

In addition to the dry powder storage at room temperature of the TFF formulated dry powder mAb, the dry powders can be encapsulated and delivered to the lung using a standard dry powder inhaler device. In certain embodiments, when tested with the Plastiape RS00 high resistance device, which is designed to provide maximum shear and aerosolization of powders at lower airflow rates, the AUG-3387 powder formulations had a fine particle fraction with greater than 50% of the powder in the 1-5 μm range, which is ideal for delivery to the deep lung of humans using a device matched to the potential for reduced lung function for mild to moderate COVID-19 patients.

In certain embodiments, the administration of AUG-3387 was determined by either intraperitoneal injection or by intratracheal insufflation of the dry powder into SARS-CoV-2 infected Syrian hamsters resulted in a dose dependent reduction of the viral load in the lung tissue of the infected hamsters. The result of this in vivo study is notable because it may be utilized to create a treatment paradigm that creates a high burden for efficacy to be demonstrated. In the study, mAb treatment of the hamsters was not initiated until 24 hours after the hamsters were infected with SARS-CoV-2 by intranasal inoculation. By contrast, sotrovimab administered by IP injection prophylactically at doses of 5 mg/kg or more when given 24- or 48-hours prior to viral infection resulted in improvement in body weight loss and decreased viral load in the lung tissue compared to control animals. Likewise, the casirivimab and imdevimab combination of mAbs administered to hamsters by IP injection 24 hours before viral inoculation (Baum et al. Science 2020 370, 1110-1115) resulted in a dose dependent viral load reduction in lung tissue. However, no change in viral load in the lung tissue was reported when casirivimab and imdevimab were administered 24 hours after viral inoculation in a manner similar to this study. For sotrovimab there was no report of therapeutic treatment resulting in reduced viral load. In certain embodiments, the demonstration that AUG-3387 administered by either IP or IT routes in a therapeutic mode resulted in a dose dependent viral load reduction in the lung tissue represents the first report of a mAb therapy that works in the hamster model in a therapeutic mode. Furthermore, the viral load reduction of the dry powder when delivered by IT insufflation represents the first report of successful reduction of viral load using inhaled delivery of a mAb therapeutic for COVID-19 disease.

In various embodiments, these data suggest that AUG-3387 is a potent mAb that has the potential to treat all known variants of SARS-CoV-2 and that the powders produced by the TFF formulation process to make room temperature stable powders has the potential to reduce the amount of mAb needed for efficacy because of the local delivery to the lung. In certain embodiments, since the TFF AUG-3387 powder does not require cold chain storage, it represents an opportunity to distribute the powder formulation globally to reduce the human cost of the COVID-19 pandemic by facilitating delivery of this therapy to locations that cannot currently utilize the therapeutic benefits because they lack cold chain distribution capabilities.

### II. Formulations of the Invention

In various embodiments, the invention generally encompasses human-derived monoclonal antibody compositions and methods comprising a dry powder formulation for neutralizing SARS-CoV-2 and known variants thereof in a patient, comprising intranasally administering to the patient a dry powder composition comprising a human-derived monoclonal antibody, AUG-3387.

In another embodiment, the invention encompasses a method for neutralizing SARS-CoV-2 virus in a patient, comprising: (a) obtaining a dry powder composition comprising a human-derived monoclonal antibody, AUG-3387 (b) suspending the dry power in pharmaceutically acceptable liquid to form a suspension; and (c) intranasally administering the suspension to the patient.

In other embodiments, the invention encompasses a dry powder formulation including a human-derived monoclonal antibody, AUG-3387, that includes less than 5% water. In embodiments, the dry powder formulation includes less than 4% water. In embodiments, the dry powder formulation includes less than 3% water. In embodiments, the dry powder formulation includes less than 2% water. In embodiments, the dry powder formulation includes less than 1% water. In embodiments, the dry powder formulation includes less than 5% water (wt/wt). In embodiments, the dry powder formulation includes less than 4% water (wt/wt). In embodiments, the dry powder formulation includes less than 3% water (wt/wt). In embodiments, the dry powder formulation includes less than 2% water (wt/wt). In embodiments, the dry powder formulation includes less than 1% water (wt/wt).

In other embodiments, the dry powder formulation including a human-derived monoclonal antibody, AUG-3387, includes about 5% water. In embodiments, the dry powder formulation includes about 4% water. In embodiments, the dry powder formulation includes about 3% water. In embodiments, the dry powder formulation includes about 2% water. In embodiments, the dry powder formulation includes about 1% water.

In other embodiments, the dry powder formulation including a human-derived monoclonal antibody, AUG-3387, includes an excipient. In embodiments, the dry powder formulation includes a plurality of different excipients. In embodiments, the excipient is a salt, sugar (saccharide), buffer, detergent, polymer, amino acid, or preservative. In embodiments, the excipient is disodium edetate, sodium chloride, sodium citrate, sodium succinate, sodium hydroxide, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, mannitol, lactose, sucrose, agarose, sorbitol, maltose, trehalose, surfactants, polysorbate 80, polysorbate 20, poloxamer 188, sorbitan monooleate, triton n101, m-cresol, benzyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco's modified eagles medium, hydrocortisone, neomycin, Von Willebrand factor, gluteraldehyde, benzethonium chloride, white petroleum, p-aminophenyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer.

In embodiments, the dry powder formulation including a human-derived monoclonal antibody, AUG-3387, includes less than 5% wt/wt of the excipient. In embodiments, the dry powder formulation includes less than 4% wt/wt of the excipient. In embodiments, the dry powder formulation includes less than 3% wt/wt of the excipient. In embodiments, the dry powder formulation includes less than 2% wt/wt of the excipient. In embodiments, the dry powder formulation includes less than 1% wt/wt of the excipient. In embodiments, the dry powder formulation includes less than 0.5% wt/wt of the excipient.

In certain embodiments, a cryoprotectant may be added to the dry powder formulation including a human-derived monoclonal antibody, AUG-3387, to protect the components present in the composition from damage during the freezing process. Examples of cryoprotectants include dimethyl sulfoxide, glycerol, monosaccharides, and polysaccharides (e.g., trehalose). A cryoprotectant may be present in amounts up to about 5% by weight.

Additionally, the solid form of the dry powder formulation is expected to be advantageous over dispersions (i.e., suspension) for stockpiling an antibody that neutralizes SARS-CoV-2, which are critical to national security and public health. For example, COVID-19 is a life-threatening disease caused by SARS-CoV-2 virus.

In another embodiment, a dry powder formulation may be composed of nano- or micro-aggregates having a particle size of less than about 200 μm (e.g., less than 10, 20, 30, 40, 50, 60, 70, 80, 90, 100, 110, 120, 130, 140, 150, 160, 170, 180, 190, 200 μm). In some embodiments, less than 5%, 4%, 3%, 2%, or 1% of the dry powder formulation upon reconstitution contains particles with a particle size greater than 100 μm.

In other embodiments, a dry formulation including a human-derived monoclonal antibody, AUG-3387, includes embodiments, examples, tables, figures, and claims. In embodiments, a dry formulation is made by a method described herein, including in aspects, embodiments, examples, tables, figures, and claims. Provided herein is a reconstituted liquid formulation comprising a dry powder formulation as described herein (including in an aspect, embodiment, example, table, figure, or claim) or a dry antibody prepared using a method as described herein (including in an aspect, embodiment, example, table, figure, or claim) and a solvent (e.g., water, buffer, solution, liquid including an excipient).

Provided in another aspect is a pharmaceutical composition including a pharmaceutically acceptable excipient and any of the compositions (e.g. antibody compositions) described herein.

The compositions described herein (including embodiments and examples) can be administered alone or can be co-administered to the patient. Coadministration is meant to include simultaneous or sequential administration of the compositions individually or in combination (more than one composition). Thus, the preparations can also be combined, when desired, with other active substances (e.g. to reduce metabolic degradation, increase immune response (e.g. adjuvants)).

Pharmaceutical compositions provided by the present embodiments include compositions wherein the active ingredient (e.g. compositions described herein, including embodiments) is contained in a therapeutically or prophylactically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., prevent infection, and/or reducing, eliminating, or slowing the progression of disease symptoms. Determination of a therapeutically or prophylactically effective amount of a composition of the embodiments is well within the capabilities of those skilled in the art, especially in light of the detailed disclosure herein.

### III. Methods of the Invention

In another embodiment, the invention encompasses a method for preparing a human-derived monoclonal antibody, AUG-3387, thin film comprising applying a liquid antibody to a freezing surface; allowing the liquid antibody to disperse and freeze on the freezing surface thereby forming an antibody thin film. In particular embodiments, the antibody is a human-derived monoclonal antibody, AUG-3387.

In other embodiments, the liquid antibody includes an excipient. In embodiments, the liquid antibody includes a plurality of different excipients. In embodiments, the excipient is a salt, sugar (saccharide), buffer, detergent, polymer, amino acid, or preservative. In embodiments, the excipient is disodium edetate, sodium chloride, sodium citrate, sodium succinate, sodium hydroxide, Sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, mannitol, lactose, sucrose, agarose, sorbitol, maltose, trehalose, surfactants, polysorbate 80, polysorbate 20, poloxamer 188, sorbitan monooleate, TRITON N101, m-cresol, benzyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco's modified eagles medium, hydrocortisone, neomycin, Von Willebrand factor, gluteraldehyde, benzethonium chloride, white petroleum, p-aminophenyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer.

In other embodiments, the liquid antibody includes less than 5% wt/vol of the excipient/liquid antibody. In embodiments, the liquid antibody includes less than 4% wt/vol of the excipient/liquid antibody. In embodiments, the liquid antibody includes less than 3% wt/vol of the excipient/liquid antibody. In embodiments, the liquid antibody includes less than 2% wt/vol of the excipient/liquid antibody. In embodiments, the liquid antibody includes less than 1% wt/vol of the excipient/liquid antibody. In embodiments, the liquid antibody includes less than 0.5% wt/vol of the excipient/liquid antibody. In certain embodiments, the liquid antibody includes about 0.1, 0.2, 0.3, 0.4, 0.5, 0.6, 0.7, 0.8, 0.9, 1, 2, 3, 4, 5, 6, 7, 8, 9, or 10% (wt/vol) of the excipient/liquid antibody. In embodiments, the liquid antibody includes less than 5% of the excipient. In embodiments, the liquid antibody includes less than 4% of the excipient. In embodiments, the liquid antibody includes less than 3% of the excipient. In embodiments, the liquid antibody includes less than 2% of the excipient. In embodiments, the liquid antibody includes less than 1% of the excipient. In embodiments, the liquid antibody includes less than 0.5% of the excipient. In embodiments, the liquid antibody includes about 0.1, 0.2, 0.3, 0.4, 0.5, 0.6, 0.7, 0.8, 0.9, 1, 2, 3, 4, 5, 6, 7, 8, 9, or 10% of the excipient.

In other embodiments, the applying includes spraying or dripping droplets of the liquid antibody. In embodiments, the vapor-liquid interface of the droplets is less than 500 cm⁻¹ area/volume. In embodiments, the vapor-liquid interface of the droplets is less than 400 cm⁻¹ area/volume. In embodiments, the vapor-liquid interface of the droplets is less than 300 cm⁻¹ area/volume. In embodiments, the vapor-liquid interface of the droplets is less than 200 cm⁻¹ area/volume. In embodiments, the vapor-liquid interface of the droplets is less than 100 cm⁻¹ area/volume. In embodiments, the vapor-liquid interface of the droplets is less than 50 cm⁻¹ area/volume. In embodiments, the vapor-liquid interface of the droplets is less than 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 cm⁻¹ area/volume.

In other embodiments, the method further includes contacting the droplets with a freezing surface having a temperature below the freezing temperature of the liquid antibody (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 degrees Celsius below the freezing temperature).

In other embodiments, the method further includes contacting the droplets with a freezing surface having a temperature differential of at least 30° C. between the droplets and the surface. In embodiments, the temperature differential is at least 40° C. between the droplets and the surface. In embodiments, the temperature differential is at least 50° C. between the droplets and the surface. In embodiments, the temperature differential is at least 60° C. between the droplets and the surface. In embodiments, the temperature differential is at least 70° C. between the droplets and the surface. In embodiments, the temperature differential is at least 80° C. between the droplets and the surface. In embodiments, the temperature differential is at least 90° C. between the droplets and the surface. In embodiments, the temperature differential between the droplets and the surface is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 degrees Celsius.

In embodiments, the antibody thin film has a thickness of less than 10 millimeters. In embodiments, the antibody thin film has a thickness of less than 8 millimeters. In embodiments, the antibody thin film has a thickness of less than 6 millimeters. In embodiments, the antibody thin film has a thickness of less than 4 millimeters. In embodiments, the antibody thin film has a thickness of less than 2 millimeters. In embodiments, the antibody thin film has a thickness of less than 1.5 millimeters. In embodiments, the antibody thin film has a thickness of less than 1.0 millimeters. In embodiments, the antibody thin film has a thickness of less than 5000 micrometers. In embodiments, the antibody thin film has a thickness of less than 4000 micrometers. In embodiments, the antibody thin film has a thickness of less than 3000 micrometers. In embodiments, the antibody thin film has a thickness of less than 2000 micrometers. In embodiments, the antibody thin film has a thickness of less than 1000 micrometers. In embodiments, the antibody thin film has a thickness of less than 500 micrometers. In embodiments, the antibody thin film has a thickness of less than 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 micrometers. In embodiments, the antibody thin film has a thickness of greater than 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 micrometers.

In embodiments, the antibody thin film has a surface area to volume ratio of between 25 and 500 cm⁻¹. In embodiments, the antibody thin film has a surface area to volume ratio of between 25 and 400 cm⁻¹. In embodiments, the antibody thin film has a surface area to volume ratio of between 25 and 300 cm⁻¹. In embodiments, the antibody thin film has a surface area to volume ratio of between 25 and 200 cm⁻¹. In embodiments, the antibody thin film has a surface area to volume ratio of between 25 and 100 cm⁻¹. In embodiments, the antibody thin film has a surface area to volume ratio of between 100 and 500 cm⁻¹. In embodiments, the antibody thin film has a surface area to volume ratio of between 200 and 500 cm⁻¹. In embodiments, the antibody thin film has a surface area to volume ratio of between 300 and 500 cm⁻¹. In embodiments, the antibody thin film has a surface area to volume ratio of between 400 and 500 cm⁻¹. In embodiments, the antibody thin film has a surface area to volume ratio of between 100 and 400 cm⁻¹. In embodiments, the antibody thin film has a surface area to volume ratio of between 200 and 300 cm⁻¹. In embodiments, the antibody thin film has a surface area to volume ratio of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 cm⁻¹. In embodiments, the antibody thin film has a surface area to volume ratio of between about 25 and about 500 cm⁻¹. In embodiments, the antibody thin film has a surface area to volume ratio of between about 25 and about 400 cm⁻¹. In embodiments, the antibody thin film has a surface area to volume ratio of between about 25 and about 300 cm⁻¹. In embodiments, the antibody thin film has a surface area to volume ratio of between about 25 and about 200 cm⁻¹. In embodiments, the antibody thin film has a surface area to volume ratio of between about 25 and about 100 cm⁻¹. In embodiments, the antibody thin film has a surface area to volume ratio of between about 100 and about 500 cm⁻¹. In embodiments, the antibody thin film has a surface area to volume ratio of between about 200 and about 500 cm⁻¹. In embodiments, the antibody thin film has a surface area to volume ratio of between about 300 and about 500 cm⁻¹. In embodiments, the antibody thin film has a surface area to volume ratio of between about 400 and about 500 cm⁻¹. In embodiments, the antibody thin film has a surface area to volume ratio of between about 100 and about 400 cm⁻¹. In embodiments, the antibody thin film has a surface area to volume ratio of between about 200 and about 300 cm⁻¹.

In embodiments, the freezing rate of the droplets is between about 10 K/second and about 10⁵ K/second. In embodiments, the freezing rate of the droplets is between about 10 K/second and about 10⁴ K/second. In embodiments, the freezing rate of the droplets is between about 10 K/second and about 10³ K/second. In embodiments, the freezing rate of the droplets is between about 10² K/second and about 10³ K/second. In embodiments, the freezing rate of the droplets is between about 50 K/second and about 5×10² K/second. In embodiments, the freezing rate of the droplets is between 10 K/second and 10⁵ K/second. In embodiments, the freezing rate of the droplets is between 10 K/second and 10⁴ K/second. In embodiments, the freezing rate of the droplets is between 10 K/second and 10³ K/second. In embodiments, the freezing rate of the droplets is between 10² K/second and 10³ K/second. In embodiments, the freezing rate of the droplets is between 50 K/second and 5×10² K/second. In embodiments, the freezing rate of the droplets is about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 K/second.

In embodiments, each of the droplets freezes upon contact with the freezing surface in less than about 50, 75, 100, 125, 150, 175, 200, 250, 500, 1,000, 2,000, 3,000, 4,000, 5,000, 7,500, or 10,000 milliseconds. In embodiments, each of the droplets freezes upon contact with the freezing surface in less than 50, 75, 100, 125, 150, 175, 200, 250, 500, 1,000, 2,000, 3,000, 4,000, 5,000, 7,500, or 10,000 milliseconds.

In embodiments, the droplets have an average diameter between about 0.1 μm and about 5 mm, between about 20 and about 24 degrees Celsius. In embodiments, the droplets have an average diameter between about 2 μm and about 4 mm, between about 20 and about 24 degrees Celsius. In embodiments, the droplets have an average diameter between about 1 4 μm and about 3 mm, between about 20 and about 24 degrees Celsius. In embodiments, the droplets have an average diameter between about 2 μm and about 2 mm, between about 20 and about 24 degrees Celsius. In embodiments, the droplets have an average diameter between about 1 μm and about 1.5 mm, between about 20 and about 24 degrees Celsius. In embodiments, the droplets have an average diameter between about 0.1 mm and about 2 mm, between about 20 and about 24 degrees Celsius. In embodiments, the droplets have an average diameter between about 0.5 mm and about 2 mm, between about 20 and about 24 degrees Celsius. In embodiments, the droplets have an average diameter between 1 mm and 2 mm, between 20 and 24 degrees Celsius. In embodiments, the droplets have an average diameter between 2 and 4 μm, between 20 and 24 degrees Celsius.

In embodiments, the method further includes removing the solvent (e.g. water or liquid) from the antibody thin film to form a dry antibody.

In embodiments, is a method of making a dry antibody from an antibody thin film (e.g. including an antibody thin film made using a method as described herein), including removing the solvent (e.g. water or liquid) from the antibody thin film to form a dry antibody. In embodiments of the methods described herein, the dry antibody is a dry antibody as described herein, including in an aspect, embodiment, example, table, figure, or claim. In embodiments, a method of making an antibody thin film or a method of making dry antibody is used to make a dry antibody as described herein, including in an aspect, embodiment, example, table, figure, or claim.

In other embodiments, the removing of the solvent includes lyophilization. In embodiments, the removing of the solvent includes lyophilization at temperatures of 20 degrees Celsius or less. In embodiments, the removing of the solvent includes lyophilization at temperatures of 25 degrees Celsius or less. In embodiments, the solvent includes lyophilization at temperatures of 40 degrees Celsius or less. In embodiments, the removing of the solvent includes lyophilization at temperatures of 50 degrees Celsius or less.

In other embodiments, the method further includes solvating the dry antibody thereby forming a reconstituted liquid antibody. A reconstituted liquid antibody may also be called a solvated dry antibody.

In other embodiments, the invention encompasses a method of making a reconstituted liquid antibody from a dry antibody (e.g. including a dry antibody made using a method as described herein), including solvating a dry antibody and thereby forming a reconstituted liquid antibody. In embodiments of the methods described herein, the dry antibody is a dry antibody as described herein, including in an aspect, embodiment, example, table, figure, or claim. In embodiments, a method of making an antibody thin film, a method of making a dry antibody, or a method of reconstituting a liquid antibody is used to make a reconstituted liquid antibody as described herein, including in an aspect, embodiment, example, table, figure, or claim.

In various embodiments, the reconstituted liquid antibody includes one or more dissolved particles. In embodiments, the particles have an average diameter of between about 10 nm and about 2 μm. In embodiments, the particles have an average diameter of between about 20 nm and about 2 μm. In embodiments, the particles have an average diameter of between about 50 nm and about 2 μm. In embodiments, the particles have an average diameter of between about 100 nm and about 2 μm. In embodiments, the particles have an average diameter of between about 200 nm and about 2 μm. In embodiments, the particles have an average diameter of between about 500 nm and about 2 μm. In embodiments, the particles have an average diameter of between about 1 μm and about 2 μm. In embodiments, the particles have an average diameter of between about 10 nm and about 1 μm. In embodiments, the particles have an average diameter of between about 10 nm and about 500 nm. In embodiments, the particles have an average diameter of between about 10 nm and about 200 nm. In embodiments, the particles have an average diameter of between about 10 nm and about 200 nm. In embodiments, the particles have an average diameter of between about 10 nm and about 100 nm. In embodiments, the particles have an average diameter of between about 10 nm and about 50 nm. In embodiments, the particles have an average diameter of between about 10 nm and about 20 nm. The solution may contain less than 5%, less than 4%, less than 3%, less than 2%, or less than 1% of undissolved particles. Even among the particles that are dissolved, the reconstituted liquid antibody may comprise a set of particles that contains less than 10%, less than 5%, less than 4%, less than 3%, less than 2%, or less than 1% of the particles with an average particle size of greater than 100 μm.

In embodiments, the particles have an average diameter of between about 20 nm and about 1 μm. In embodiments, the particles have an average diameter of between about 50 nm and about 500 nm. In embodiments, the particles have an average diameter of between about 100 nm and about 500 nm. In embodiments, the particles have an average diameter of between about 100 nm and about 200 nm. In embodiments, the reconstituted liquid antibody includes particles, wherein the particles include the antigenic protein adsorbed to the aluminum adjuvant. In embodiments, the particles have an average diameter of between 10 nm and 2 μm. In embodiments, the particles have an average diameter of between 20 nm and 2 μm. In embodiments, the particles have an average diameter of between 50 nm and 2 μm. In embodiments, the particles have an average diameter of between 100 nm and 2 μm. In embodiments, the particles have an average diameter of between 200 nm and 2 μm. In embodiments, the particles have an average diameter of between 500 nm and 2 μm. In embodiments, the particles have an average diameter of between 1 μm and 2 μm. In embodiments, the particles have an average diameter of between 10 nm and 1 μm. In embodiments, the particles have an average diameter of between 10 nm and 500 nm. In embodiments, the particles have an average diameter of between 10 nm and 200 nm. In embodiments, the particles have an average diameter of between 10 nm and 200 nm. In embodiments, the particles have an average diameter of between 10 nm and 100 nm. In embodiments, the particles have an average diameter of between 10 nm and 50 nm. In embodiments, the particles have an average diameter of between 10 nm and 20 nm. In embodiments, the particles have an average diameter of between 20 nm and 1 μm. In embodiments, the particles have an average diameter of between 50 nm and 500 nm. In embodiments, the particles have an average diameter of between 100 nm and 500 nm. In embodiments, the particles have an average diameter of between 100 nm and 200 nm. In embodiments, the particles are non-crystalline. In embodiments, the particles are amorphous.

In other embodiments, the particles have an average diameter of between about 1 μm and about 50 μm. In embodiments, the particles have an average diameter of between about 10 μm and about 50 μm. In embodiments, the particles have an average diameter of between about 20 μm and about 50 μm. In embodiments, the particles have an average diameter of between about 30 μm and about 50 μm. In embodiments, the particles have an average diameter of between about 40 μm and about 50 μm. In embodiments, the particles have an average diameter of between about 10 μm and about 40 μm. In embodiments, the particles have an average diameter of between about 10 μm and about 30 μm. In embodiments, the particles have an average diameter of between about 10 μm and about 20 μm. In embodiments, the particles have an average diameter of between about 1 μm and about 10 μm. In embodiments, the particles have an average diameter of between 1 μm and 50 μm. In embodiments, the particles have an average diameter of between 10 μm and 50 μm. In embodiments, the particles have an average diameter of between 20 μm and 50 μm. In embodiments, the particles have an average diameter of between 30 μm and 50 μm. In embodiments, the particles have an average diameter of between 40 μm and 50 μm. In embodiments, the particles have an average diameter of between 10 μm and 40 μm. In embodiments, the particles have an average diameter of between 10 μm and 30 μm. In embodiments, the particles have an average diameter of between 10 μm and 20 μm. In embodiments, the particles have an average diameter of between 1 μm and 10 μm. In embodiments, the particles have an average diameter of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 μm. In embodiments, the particles have an average diameter of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 μm.

In embodiments, the solvating of the dry antibody is at least one day after preparing the dry antibody from the liquid antibody (e.g. the dry antibody is stored for at least one day). In embodiments, the solvating of the dry antibody is at least two days after preparing the dry antibody from the liquid antibody (e.g. the dry antibody is stored for at least two days). In embodiments, the solvating of the dry antibody is at least three days after preparing the dry antibody from the liquid antibody (e.g. the dry antibody is stored for at least three days). In embodiments, the solvating of the dry antibody is at least one week after preparing the dry antibody from the liquid antibody (e.g. the dry antibody is stored for at least one week). In embodiments, the solvating of the dry antibody is at least two weeks after preparing the dry antibody from the liquid antibody (e.g. the dry antibody is stored for at least two weeks). In embodiments, the solvating of the dry antibody is at least one month after preparing the dry antibody from the liquid antibody (e.g., the dry antibody is stored for at least one month). In embodiments, the solvating of the dry antibody is at least two months after preparing the dry antibody from the liquid antibody (e.g. the dry antibody is stored for at least two months). In embodiments, the solvating of the dry antibody is at least three months after preparing the dry antibody from the liquid antibody (e.g. the dry antibody is stored for at least three months). In embodiments, the solvating of the dry antibody is at least six months after preparing the dry antibody from the liquid antibody (e.g. the dry antibody is stored for at least six months). In embodiments, the solvating of the dry antibody is at least one year after preparing the dry antibody from the liquid antibody (e.g. the dry antibody is stored for at least one year). In embodiments, the solvating of the dry antibody is at least two years after preparing the dry antibody from the liquid antibody (e.g. the dry antibody is stored for at least two years). In embodiments, the solvating of the dry antibody is at least three years after preparing the dry antibody from the liquid antibody (e.g. the dry antibody is stored for at least three years). In embodiments, the solvating of the dry antibody is at least five years after preparing the dry antibody from the liquid antibody (e.g. the dry antibody is stored for at least five years). In embodiments, the solvating of the dry antibody is at least ten years after preparing the dry antibody from the liquid antibody (e.g. the dry antibody is stored for at least ten years).

In embodiments, prior to the solvating of the dry antibody, the dry antibody is stored at about 4 degrees Celsius for at least 99% of the time. In embodiments, prior to the solvating of the dry antibody, the dry antibody is stored at less than 4 degrees Celsius for at least 99% of the time. In embodiments, prior to the solvating of the dry antibody, the dry antibody is stored at less than 0 degrees Celsius for at least 99% of the time. In embodiments, prior to the solvating of the dry antibody, the dry antibody is stored at less than −20 degrees Celsius for at least 99% of the time. In embodiments, prior to the solvating of the dry antibody, the dry antibody is stored at about −20 degrees Celsius for at least 99% of the time. In embodiments, prior to the solvating of the dry antibody, the dry antibody is stored at less than ˜80 degrees Celsius for at least 99% of the time. In embodiments, prior to the solvating of the dry antibody, the dry antibody is stored at about −80 degrees Celsius for at least 99% of the time. In embodiments, prior to the solvating of the dry antibody, the dry antibody is stored at ambient temperatures (e.g. room temperature). In embodiments, prior to the solvating of the dry antibody, the dry antibody is stored at between 20 and 24 degrees Celsius for at least 99% of the time. In embodiments, prior to the solvating of the dry antibody, the dry antibody is stored at between 4 and 24 degrees Celsius for at least 99% of the time. In embodiments, prior to the solvating of the dry antibody, the dry antibody is stored at between 0 and 24 degrees Celsius for at least 99% of the time. In embodiments, prior to the solvating of the dry antibody, the dry antibody is stored at between 4 and 40 degrees Celsius for at least 99% of the time. In embodiments, prior to the solvating of the dry antibody, the dry antibody is stored at between 0 and 40 degrees Celsius for at least 99% of the time.

In embodiments, prior to the solvating of the dry antibody, the dry antibody is stored at about 4 degrees Celsius for at least 90% of the time. In embodiments, prior to the solvating of the dry antibody, the dry antibody is stored at less than 4 degrees Celsius for at least 90% of the time. In embodiments, prior to the solvating of the dry antibody, the dry antibody is stored at less than 0 degrees Celsius for at least 90% of the time. In embodiments, prior to the solvating of the dry antibody, the dry antibody is stored at less than −20 degrees Celsius for at least 90% of the time. In embodiments, prior to the solvating of the dry antibody, the dry antibody is stored at between 20 and 24 degrees Celsius for at least 90% of the time. In embodiments, prior to the solvating of the dry antibody, the dry antibody is stored at between 4 and 24 degrees Celsius for at least 90% of the time. In embodiments, prior to the solvating of the dry antibody, the dry antibody is stored at between 0 and 24 degrees Celsius for at least 90% of the time. In embodiments, prior to the solvating of the dry antibody, the dry antibody is stored at between 4 and 40 degrees Celsius for at least 90% of the time. In embodiments, prior to the solvating of the dry antibody, the dry antibody is stored at between 0 and 40 degrees Celsius for at least 90% of the time.

In other embodiments, upon solvating the dry antibody the resulting reconstituted liquid antibody remains homogeneous. As used in reference to the status of a reconstituted liquid antibody, the term “homogenous” refers to a lack of a significant amount of aggregation and/or precipitation forming, such that the reconstituted liquid antibody does not include solid matter that is not evenly dispersed (e.g. solid matter visible to the naked eye, solid matter that settles in the liquid, solid matter that was not apparent in a liquid antibody prior to formation of the dry antibody and reconstitution, precipitate that was not present in the liquid antibody prior to formation of the dry antibody). In embodiments, upon solvating the dry antibody the resulting reconstituted liquid antibody remains homogeneous for at least one day. In embodiments, upon solvating the dry antibody the resulting reconstituted liquid antibody remains homogeneous for at least two days. In embodiments, upon solvating the dry antibody the resulting reconstituted liquid antibody remains homogeneous for at least three days. In embodiments, upon solvating the dry antibody the resulting reconstituted liquid antibody remains homogeneous for at least one week. In embodiments, upon solvating the dry antibody the resulting reconstituted liquid antibody remains homogeneous for at least two weeks. In embodiments, upon solvating the dry antibody the resulting reconstituted liquid antibody remains homogeneous for at least one month. In embodiments, upon solvating the dry antibody the resulting reconstituted liquid antibody remains homogeneous for at least three months. In embodiments, upon solvating the dry antibody the resulting reconstituted liquid antibody remains homogeneous for at least six months. In embodiments, upon solvating the dry antibody the resulting reconstituted liquid antibody remains homogeneous for at least one year. In embodiments, upon solvating the dry antibody the resulting reconstituted liquid antibody does not form a precipitate (e.g. solid matter visible to the naked eye, solid matter that settles in the liquid, solid matter that was not apparent in a liquid antibody prior to formation of the dry antibody and reconstitution, precipitate that was not present in the liquid antibody prior to formation of the dry antibody). In embodiments, upon solvating the dry antibody the resulting reconstituted liquid antibody does not form a precipitate for at least one day. In embodiments, upon solvating the dry antibody the resulting reconstituted liquid antibody does not form a precipitate for at least two days. In embodiments, upon solvating the dry antibody the resulting reconstituted liquid antibody does not form a precipitate for at least three days. In embodiments, upon solvating the dry antibody the resulting reconstituted liquid antibody does not form a precipitate for at least one week. In embodiments, upon solvating the dry antibody the resulting reconstituted liquid antibody does not form a precipitate for at least two weeks. In embodiments, upon solvating the dry antibody the resulting reconstituted liquid antibody does not form a precipitate for at least one month. In embodiments, upon solvating the dry antibody the resulting reconstituted liquid antibody does not form a precipitate for at least three months. In embodiments, upon solvating the dry antibody the resulting reconstituted liquid antibody does not form a precipitate for at least six months. In embodiments, upon solvating the dry antibody the resulting reconstituted liquid antibody does not form a precipitate for at least one year. In embodiments, the precipitate includes particles having an average diameter greater than 100 μm. In embodiments, the precipitate includes particles having an average diameter greater than 200 μm. In embodiments, the precipitate includes particles having an average diameter greater than 300 μm. In embodiments, the precipitate includes particles having an average diameter greater than 400 μm. In embodiments, the precipitate includes particles having an average diameter greater than 500 μm. In embodiments, the precipitate includes particles having an average diameter greater than 600 μm. In embodiments, the precipitate includes particles having an average diameter greater than 700 μm. In embodiments, the precipitate includes particles having an average diameter greater than 800 μm. In embodiments, the precipitate includes particles having an average diameter greater than 900 μm. In embodiments, the precipitate includes particles having an average diameter greater than 1000 μm.

In embodiments, the precipitate includes particles having an average diameter greater than about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μm.

In embodiments, the precipitate includes particles having an average diameter of about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μm.

In embodiments, the liquid antibody includes the antibody, AUG-3387. In other embodiments, the liquid antibody is a commercially available antibody. In embodiments, the liquid antibody has received market approval from the U.S. FDA or the corresponding authority in another country. In embodiments, the liquid antibody is an antibody for the treatment of COVID-19 or related symptoms. In embodiments, the liquid antibody is any antibody for the treatment of infection by SARS-CoV-2. In embodiments, the liquid antibody includes AUG-3387 and another component (e.g., an excipient).

In another embodiment is provided a method of treating a disease in a patient in need of such treatment, the method including administering a therapeutically effective amount of a solvated dry antibody as described herein (e.g. in an aspect, embodiment, example, table, figure, or claims) (e.g. a reconstituted liquid antibody as described herein) to the patient.

In another embodiment is provided a method of treating a coronavirus associated disease in a patient in need of such treatment, the method including administering a therapeutically effective amount of dry antibody as described herein (e.g., in an aspect, embodiment, example, table, figure, or claims) (e.g. a reconstituted liquid antibody as described herein) to the patient.

In embodiments, the disease is COVID-19.

In embodiments, the dry antibody is administered by inhalation, intradermally, or orally. In embodiments, the dry antibody is administered through the nasal mucosa, bronchoalveolar mucosa, or gastrointestinal mucosa.

In other embodiments, the method is a method described herein, including in an aspect, embodiment, example, table, figure, or claim. Provided herein is a method of preparing a dry antibody including a method of preparing an antibody thin film as described herein (including in an aspect, embodiment, example, table, figure, or claim) and a method of removing a solvent from an antibody thin film as described herein (including in an aspect, embodiment, example, table, figure, or claim). Provided herein is a method of preparing a reconstituted dry antibody including a method of preparing a dry antibody as described herein (including in an aspect, embodiment, example, table, figure, or claim), a method of preparing an antibody thin film as described herein (including in an aspect, embodiment, example, table, figure, or claim) and a method of removing a solvent from an antibody thin film as described herein (including in an aspect, embodiment, example, table, figure, or claim).

In another embodiment, the invention encompasses a method to form a powder antibody. An aqueous antibody composition is first frozen to form a frozen antibody composition, then the frozen water is removed to form the antibody powder. A fast freezing process is used to form the frozen antibody composition. A fast freezing process, as used herein, is a process that can freeze a thin film of liquid (less than about 500 microns) in a time of less than or equal to one second. Examples of fast freezing processes that may be used include thin film freezing (TFF), spray freeze-drying (SFD), or spray freezing into liquids (SFL). In the TFF process liquid droplets fall from a given height and impact, spread, and freeze on a cooled solid substrate. Typically, the substrate is a metal drum that is cooled to below 250° K, or below 200° K or below 150° K. On impact the droplets that are deformed into thin films freeze in a time of between about 70 ms and 1000 ms. The frozen thin films may be removed from the substrate by a stainless steel blade mounted along the rotating drum surface. The frozen thin films are collected in liquid nitrogen to maintain in the frozen state. Further details regarding thin film freezing processes may be found in the paper to Engstrom et al. “Formation of Stable Submicron Protein Particles by Thin Film Freezing” Pharmaceutical Research, Vol. 25, No. 6, June 2008, 1334-1346, which is incorporated herein by reference.

Water (e.g., frozen water) is removed from the frozen antibody composition to produce an antibody powder. Water (e.g. frozen water) may be removed by a lyophilization process or a freeze-drying process. Water may also be removed by an atmospheric freeze-drying process.

The resulting antibody powder can be readily reconstituted to form a stable dispersion without significant loss of stability or activity. The antibody powder may be transported and stored in a wide range of temperatures without concern of accidental exposure to freezing conditions. In addition, the antibody powder may also be stored at room temperature, which will potentially decrease the costs of antibodies. In fact, it is generally less costly to transport dry solid powder than liquid.

Currently human antibodies (e.g. marketed and/or approved human antibodies, such as FDA approved human antibodies) are typically administered by needle-syringe-based injection or infusion. It would be beneficial to patients and the healthcare system if the antibodies were administered non-invasively without needles. The antibody powder can potentially be administered by an alternative route such as, but not limited to, inhalation as a dried powder, intradermally using a solid jet injection device (e.g., powder jet injector), orally or nasally by inhalation, orally in tablets or capsules, or buccally in buccal tablets or films. The above-mentioned routes of administration are not only more convenient and friendly to patients, but more importantly they can enable the induction of mucosal responses. Functional antibodies in the mucosal secretion (e.g., nasal mucus, bronchoalveolar mucus, or the gastrointestinal mucus) can effectively neutralize virus even before they enter the host.

Described herein are compositions and methods for preparing an antibody thin film or a dry antibody by spraying or dripping droplets of a liquid antibody such that the liquid antibody is exposed to a vapor-liquid interface of less than 500 cm⁻¹ area/volume (e.g., less than 50, 100, 150, 200, 250, 300, 400 cm⁻¹ area/volume) and contacting the droplet with a freezing surface having a temperature lower than the freezing temperature of the liquid antibody (e.g. has a temperature differential of at least 30° C. between the droplet and the surface), wherein the surface freezes the droplet into a thin film with a thickness of less than 500 micrometers (e.g., greater than 450, 400, 350, 300, 250, 200, 150, 100, or 50 micrometers) and a surface area to volume between 25 to 500 cm⁻¹. In embodiments, the method may further include the step of removing the liquid (e.g. solvent, water) from the frozen material to form a dry antibody (e.g., particles). In embodiments, the droplets freeze upon contact with the surface in less than 50, 75, 100, 125, 150, 175, 200, 250, 500, 1,000, 2,000, 3,000, 4,000, 5,000, 7,500, or 10,000 milliseconds. In embodiments, the droplets freeze upon contact with the surface in less than 50 or 150 milliseconds. In embodiments, the droplet has a diameter between 0.1 μm and 5 mm at room temperature. In embodiments, the droplet forms a thin film on the freezing surface of between 50 and 5000 micrometers in thickness. In embodiments, the droplets have a cooling rate of between 50-250 K/s. In embodiments, the particles of the dry antibody, after liquid (e.g. solvent or water) removal, have a surface area of at least 10, 15, 25, 50, 75, 100, 125, 150 or 200 m²/gr (e.g. surface area of 10, 15, 25, 50, 75, 100, 125, 150 or 200 m²/gr).

In embodiments, the droplets may be delivered to the cold or freezing surface in a variety of manners and configurations. In embodiments, the droplets may be delivered in parallel, in series, at the center, middle or periphery or a platen, platter, plate, roller, conveyor surface. In embodiments, the freezing or cold surface may be a roller, a belt, a solid surface, circular, cylindrical, conical, oval and the like that permit for the droplet to freeze. For a continuous process a belt, platen, plate or roller may be particularly useful. In embodiments, the frozen droplets may form beads, strings, films or lines of frozen liquid antibody. In embodiments, the effective ingredient is removed from the surface with a scraper, wire, ultrasound or other mechanical separator prior to the lyophilization process. Once the material is removed from the surface of the belt, platen, roller or plate the surface is free to receive additional material.

In embodiments, the surface is cooled by a cryogenic solid, a cryogenic gas, a cryogenic liquid or a heat transfer fluid capable of reaching cryogenic temperatures or temperatures below the freezing point of the liquid antibody (e.g. at least 30° C. less than the temperature of the droplet). In embodiments, the liquid antibody further includes one or more excipients selected from sugars, phospholipids, surfactants, polymeric surfactants, polymers, including copolymers and homopolymers and biopolymers, dispersion aids. In embodiments, the temperature differential between the droplet and the surface is at least 50° C. In embodiments, the excipients or stabilizers that can be included in the liquid antibodies that are to be frozen as described herein include: cryoprotectants, lyoprotectants, surfactants, fillers, stabilizers, polymers, antioxidants and absorption enhancers. Specific nonlimiting examples of excipients that may be included in the antibodies described herein include: sucrose, trehaolose, Span 80, Tween 80, Brij 35, Brij 98, Pluronic, sucroester 7, sucroester 11, sucroester 15, sodium lauryl sulfate, oleic acid, laureth-9, laureth-8, lauric acid, vitamin E TPGS, Gelucire 50/13, Gelucire 53/10, Labrafil, dipalmitoyl phosphadityl choline, glycolic acid and salts, deoxycholic acid and salts, sodium fusidate, cyclodextrins, polyethylene glycols, labrasol, polyvinyl alcohols, polyvinyl pyrrolidones and tyloxapol.

In other embodiments, the method may further include the step of removing the liquid (e.g. solvent or water) from the frozen liquid antibody to form a dry antibody. In embodiments, the solvent further includes at least one or more excipient or stabilizers selected from, e.g., sugars, phospholipids, surfactants, polymeric surfactants, vesicles, polymers, including copolymers and homopolymers and biopolymers, dispersion aids, and serum albumin. In embodiments, the temperature differential between the solvent and the surface is at least 50° C.

In other embodiments, the resulting powder can be used without further dispersion into an aqueous medium. In other embodiments, the resulting powder can be redispersed into a suitable aqueous medium such as saline, buffered saline, water, buffered aqueous media, solutions of amino acids, solutions of vitamins, solutions of carbohydrates, or the like, as well as combinations of any two or more thereof, to obtain a suspension that can be administered to mammals (e.g. humans).

In other embodiments, is described a single-step, single-vial method for preparing an antibody thin film or dry antibody by reducing the temperature of a vial wherein the vial has a temperature below the freezing temperature of a liquid antibody (e.g. a temperature differential of at least 30° C. between the liquid antibody and the vial) and spraying or dripping droplets of a liquid antibody directly into the vial such that the liquid antibody is exposed to a vapor-liquid interface of less than 500 cm⁻¹ area/volume, wherein the surface freezes the droplet into a thin film with a thickness of less than 5000 micrometers and a surface area to volume between 25 to 500 cm⁻¹. In embodiments, the droplets freeze upon contact with the surface in less than about 50, 75, 100, 125, 150, 175, 200, 250, 500, 1,000 or 2,000 milliseconds (e.g. in about 50, 75, 100, 125, 150, 175, 200, 250, 500, 1,000 or 2,000 milliseconds), and may freeze upon contact with the surface in about 50 or 150 to 500 milliseconds.

In embodiments, a droplet has a diameter between 0.1 μm and 5 mm at room temperature (e.g. a diameter between 2 and 4 mm at room temperature). In embodiments, the droplet forms a thin film on the surface of between 50 micrometers and 5000 micrometers in thickness. In embodiments, the droplets have a cooling rate of between 50-250 K/s. In embodiments, the vial may be cooled by a cryogenic solid, a cryogenic gas, a cryogenic liquid, a freezing fluid, a freezing gas, a freezing solid, a heat exchanger, or a heat transfer fluid capable of reaching cryogenic temperatures or temperatures below the freezing point of the liquid antibody. In embodiments, the vial may be rotated as the spraying or droplets are delivered to permit the layering or one or more layers of the liquid antibody. In embodiments, the vial and the liquid antibody are pre-sterilized prior to spraying or dripping. In embodiments, the step of spraying or dripping is repeated to overlay one or more thin films on top of each other to fill the vial to any desired level up to totally full.

### Examples

1.1 Antibody Isolation

For this study, patients were profiled with low disease burden, rather than severely affected individuals. It was believed that asymptomatic or weakly symptomatic patients might have had previous exposure to a related antigen (e.g., other coronaviruses), providing a breadth of antigenic coverage and driving their resistance to Covid-19. Informed consent was obtained from all patients, and all patient samples were collected after a full recovery from illness and under IRB approval. A total of seven study subjects were profiled for the presence of SARS-CoV-2 antibodies. See FIG. 1.

From each sample, plasma was separated from PBMC's and red blood cells by Ficoll density gradient centrifugation. Additionally, approximately 100k B cells were extracted from whole blood (RosetteSep) and seeded directly, or terminally differentiated into plasma cells and then seeded into SingleCyte® assay plates with up to 4 different optically encoded SARS-CoV-2 antigens or controls. A fluorescently labelled anti-human IgG/IgA/IgM secondary antibody was also added to each well. After a 24 hour incubation, antigen specific cells were identified by their signature reaction-diffusion pattern. For comparison, Alexa Fluor 488 stained SARS-CoV-2 antigen was used as a staining agent for single cell sorting of antigen reactive memory B cells into 96 well plates on a Sony SH-800 Cell Sorter. Cells specific to any SARS-CoV or SARS-CoV-2 antigen were retrieved and sequenced.

The sequences were analyzed for unique clonotypes based on CDR3 sequences of heavy and light chains, then unique clonotypes were designed into DNA fragments for cloning through our AbWorks™ automated clone design software. The fragments were cloned as ScFv's for expression in E. coli, or as full heavy and light chains in mammalian expression vectors for tandem transfection into Expi293T.

SingleCyte is a programmable single cell imaging cytometer and sorter that selects cells based on temporal microscopy. For screening of secreted antibody proteins, assay plates contain a multiplexed panel of antigens in the form of conjugated beads or antigen presenting cells and a secondary antibody in solution. Antibodies from secreted cells bind proximal antigens and become physically constrained near the secreting cell. Fluorescent secondary antibody enables visualization of secreted antibody concentration gradients based on fluorescence over time, and optically encoded antigen beads enables deconvolution of target antigens. Assays are performed in standard open well SBS footprint microplates and are user programmable. A custom nanoliter volume micropipette enables isolation of single cells, and a robotic arm carries receiver plates for high throughput single cell retrieval.

The instrument typically works by first raster scanning each well. Antigen specific cells with any antigen reactivity are identified by processing images with a convolutional neural network trained on a set of manually curated images. The microscope performs multispectral high resolution imaging of each positive cell. Images are masked into regions by the optical characteristics of proximal beads and a confidence score is ascribed to each cell-antigen interaction. Cells are then picked and placed into receiver plates and after images are taken to ensure proper aspiration of target cells. The information for each run and output metrics for each cell (including both source and destination locations) are saved to a database for recall in a user interface and for downstream processing steps.

1.2. Antibody Characterization—Target Binding Characterization

SARS-CoV-2 S1 and RBD proteins, and various other antigens and controls, were covalently coupled to Luminex MagPlex magnetic microspheres for assay binding with a Luminex 200 instrument. The following antigens were conjugated: B.1.1.17 (Alpha) S1, B.1.1.28 (Gamma) S1+S2, 20H/501Y.V2 (Beta) S1, B.1.617 (Kappa) RBD, B.1.617.2 (Delta) RBD, S1+S2 S494P, S1+S2 V483A, S1+S2 R683A+R685A+F817P+A892P+A899P+A942P+K986P+V987P, S1+S2 G485S, S1+S2 D614G, S1+S2 E484K, S1+S2 D614G+V445I+H655Y+E583D, S1+S2 L452R+T478K. Each antigen was conjugated with the xMAP conjugation kit at ratio of 5 μg protein to 1 million beads. Assays were performed in multiplex, with each spectrally encoded bead having a separate antigen and run together in a single well. Antibody was titrated over therapeutically relevant concentrations, mixed with the beads, washed twice, labelled with a secondary antibody, washed twice and run on the instrument. Dry powder versions of antibodies were resuspended in water before dilution for assay.

The ScFv version of AUG-3387, AUG-3705, was run on a Carterra LSA instrument at multiple concentrations for determination of the single domain affinity against Wuhan SARS-CoV-2 S1 and RBD. AUG-3705 was attached to the LSA flow cell via interaction with its V5 tag and a surface bound anti-V5 antibody. Wuhan-1 RBD was delivered to the flow cell at concentrations of 2.06 nM, 6.17 nM, 18.5 nM and 56 nM for calculation of Kd.

1.3. Pseudo-Neutralization Assay—ACE-2 Expressing HEK293T Cell Line Construction

An ACE2 expressing HEK293T cell line (“LentiX ACE2.S4”) was constructed by packaging pCMV-AC-GFP (Origene) into lentivirus and transducing HEK293T's. The cells were enriched 4 times until 97% of the cells showed signal above the negative control as read out by staining with anti-ACE-2 and secondary antibodies. On average enriched ACE2-HEK293T's had 50× the signal of non-transduced cells.

1.4. Pseudo-Neutralization Assay—SARS-CoV-2 Pseudovirus with Powder Formulation and Soluble AUG-3387

Two days prior to infection, LentiX ACE2.S4 cells were grown to 85% confluency, then seeded in a 96-well plate at 15k cells/well in 50 μL media per well and held at 37° C. in 5% CO₂ until infection. Antibody mixes were created prior to infection by performing a 128-fold serial dilution starting at 40 μg/μL. Lyophilized powders of AUG-3387 and negative control V5 Tag monoclonal antibody were seeded in triplicate, and soluble AUG-3387 was seeded in duplicate. SARS-CoV-2 pseudovirus (Genscript) was diluted in DMEM complete media to an IFU of 3.2e7/mL, and 100 μL of virus solution was mixed with 100 μL of diluted antibody. The virus/antibody mix was incubated for 60 minutes at 37° C. in 5% CO₂. Following incubation, 50 μL of each pseudovirus/antibody condition mix was added to each well of seeded cells. Additional controls included cells only, and cells with virus only. After 48 hours, the plate was removed and equilibrated at room temperature for 10 minutes, and 60 μL of the supernatant was removed. 50 μL of Promega's Bright-Glo Luciferase assay reagent was added to each well of the infected cells. The cells then were incubated at room temperature for 3 minutes, and luminescence was measured with a Tecan Spark microplate reader with a 1 second integration time.

1.5. Pseudo-Neutralization Assay—SARS-CoV-2 Pseudovirus, Delta Variant (B.1.617.2) with Soluble AUG-3387

Two days prior to infection, LentiX ACE2.S4 cells were grown to 85% confluency, then seeded in a 96-well plate at 15k cells/well in 50 μL media per well and held at 37° C. in 5% CO₂ until infection. Antibody mixes were created prior to infection by performing a 128-fold serial dilution starting at 160 μg/μL. AUG-3387 and negative control V5 Tag monoclonal antibody were seeded in triplicate. SARS-CoV-2 Delta Variant pseudovirus (eEnzyme) was diluted 1:2 in DMEM complete media to a pseudoviral particle concentration of 5e7/mL, and 200 μL of virus solution was mixed with 200 μL of diluted antibody. The virus/antibody mix was incubated for 60 minutes at 37° C. in 5% CO₂. Following incubation, 100 μL of each pseudovirus/antibody condition mix was added to each well of seeded cells. Additional controls included cells only, and cells with virus only. After 48 hours, the plate was removed and equilibrated at room temperature for 10 minutes, and 100 μL of the supernatant was removed. 50 μL of Promega's Bright-Glo Luciferase assay reagent was added to each well of the infected cells. The cells then were incubated at room temperature for 3 minutes, and luminescence was measured with a Tecan Spark microplate reader with a 1 second integration time.

1.6. SARS-Cov-2 Neutralization Assay

Two days prior to infection, Calu-3 cells were grown to confluency, then seeded at 40k cells in 100 μL media per well. Antibody was titrated in D10 media. For each antibody condition, 40 μL SARS-CoV-2 virus at a target MOI of 0.05 was added and the mixture was incubated at 37° C. for 60 minutes. Media was removed from the seeded cells and replaced with a final volume of 50 μL of antibody/virus mix. Cells with antibody and virus were incubated at 37° C. in 5% CO₂ for 30 minutes. The virus/antibody mix was removed, the cells washed with 75 μL PBS, 75 μL of media was added to each well, and then 75 μL of titrated antibody in media was added to yield a final volume of 150 μL.

After 24 hours, 50 μL of the supernatant was removed for TCID50 assays. Vero E6 cells were seeded at 10k cells in 100 μL per well. Infected cell culture supernatant was diluted with 950 μL D10 media, and then serial diluted. 50 μL of each dilution was added to 8 wells of Vero E6. After 72 hours, wells with complete cytopathic effect were counted.

After 96 hours, 100 μL CellTiterGlo reagent was added to each well of the infected cells to assay for live cells. Following incubation of CTG reagent for 20 minutes, luminescence was measured with a Spectramax 1 L with is integration time.

1.7. Preparation of Thin Film Freezing (TFF) Composition

In the preparation of the solutions for TFF manufacturing, AUG-3387 was combined with a mannitol/leucine or trehalose/leucine. The solution was applied as drops onto a rotating cryogenically cooled drum cooled to −70° C. The frozen solids were collected and stored in a −80° C. freezer before lyophilization. The lyophilization was performed in ana SP VirTis Advantage Pro shelf lyophilizer (SP Industries, Inc., Warminster, Pa., USA). The primary drying process was at −40° C. for 20 h, and then, the temperature was linearly increased to 25° C. over 20 h, followed by secondary drying at 25° C. for 20 h. The pressure was maintained at less than 100 mTorr during the lyophilization process.

1.8. Aerodynamic Particle Size Distribution Analysis

About three milligrams of AUG-3387 mAb dry powder was loaded into size #3 hydroxypropyl methylcellulose (HPMC) capsules (Vcaps® plus, Capsugel®, Lonza, Morristown, N.J., USA). The aerodynamic properties of the powder were evaluated using a Next Generation Impactor (NGI) (MSP Corporation, Shoreview, Minn., USA) connected to a High-Capacity Pump (model HCP5, Copley Scientific, Nottingham, UK) and a Critical Flow Controller (model TPK 2000, Copley Scientific, Nottingham, UK). The dry powder inhaler device RS00 (Plastiape®, Osnago, Italy) was used for dispersing the powder through the USP induction port with a total flow rate of 60 L/min for 4 s per each actuation corresponding to a 4 kPa pressure drop across the device and a total flow volume of 4 L. To avoid particle bounce, a solution of polysorbate 20 in methanol at 1.5% (w/v) was applied and dried onto the NGI collection plates to coat their surface. The pre-separator was not used in this analysis. After dispersal, the powder was extracted from the stages using water. Then, the samples were analyzed by using HPLC-ELSD to determine the content of sugar or sugar alcohol as described below. The analysis was conducted three times (n=3). The NGI results were analyzed using the Copley Inhaler Testing Data Analysis Software 3.10 (CITDAS) (Copley Scientific, Nottingham, UK). CITDAS provided the calculation for mass median aerodynamic diameter (MMAD), total dose per shot, calculated delivered dose, fine particle dose, fine particle fraction of delivered dose (FPF %, delivered) and recovered dose (FPF %, recovered), and geometric standard deviation (GSD).

1.9. Efficacy of AUG-3387 in and In Vivo Model of SARS-CoV-2 Infected Syrian Hamsters

An in vivo efficacy study was performed with male Syrian Hamsters (Mesocricetus auratus) approximately 9 weeks of age with a weight range of 110-134 g, at time of randomization, were sourced from Charles River Laboratory. Animal work was performed at Lovelace Biomedical Research Institute (LBRI), with approval from the Institutional Animal Care and Use Committee (IACUC) and within Animal Biosafety Level 3 (ABSL3) containment. Hamsters were singly housed in filter-topped cage systems and were supplied with a certified diet, filtered municipal water, and dietary and environmental enrichment. The challenge study design is detailed in Table 1. Animals were assigned to groups using a stratified (body weight) randomization procedure. Animals were anesthetized and swabs of nasal passages were collected by placing the nasal swab (0.5 mm diameter Ultrafine Micro Plasdent swabs) 1-3 mm into the nare and swabbing. Lung and nasal swab (in Trizol) samples were stored at −80° C. prior to analysis. All animals were euthanized with an euthanasia solution consisting of 390 mg of sodium pentobarbital and 50 mg of phenytoin per mL.

TABLE 1
Group Designations of animals in the efficacy study

| | | | | | | |
|---|---|---|---|---|---|---|
| | Group | Challenge | | | Number of | |
| Group | Description | Day | Dose | Route/Frequency | animals | Study Endpoints |

| | | | | | | | |
|---|---|---|---|---|---|---|---|
| 1 | Vehicle | Day 0 | 0.0 | | IP; once on Day 1 | 6 | Daily Clinical |
| | Control | | | | | | Observations Twice |
| 2 | mAb-1 | Day 0 | 1.0 | mg/kg | IT; once daily on | 6 | Daily |
| | High dose | | | | Days 1, 2, 3 | | Body Weights Daily |
| 3 | mAb-1 | Day 0 | 0.33 | mg/kg | IT; once daily on | 6 | Nasal swabs on Days |
| | Low dose | | | | Days 1, 2, 3 | | 1 and 5 for viral load |
| 4 | mAb- | Day 0 | 10.0 | mg/kg | IP; once on Day 1 | 6 | Necropsy on day 5 |
| | solution | | | | | | for viral load of |
| | High dose | | | | | | lung tissue |
| 5 | mAb- | Day 0 | 3.3 | mg/kg | IP; once on Day 1 | 6 | and Histopathology |

| | |
|---|---|
| solution | of lungs |
| Low dose | |

1.11 Viral Challenge

SARS-CoV-2, isolate USA-WA1/2020, was sourced from WRCEVA and propagated in Vero E6 African Green Monkey kidney cells (BEI, catalog #N596) in Dulbecco's Modified Eagle Medium supplemented with 1% HEPES, 10% FBS, 100 IU/mL Penicillin G and 100 μg/mL Streptomycin. Stocks were stored in a BSL-3 compliant facility at −80° C. prior to challenge. Stock vials of virus were thawed the day of challenge, diluted as necessary, and stored on wet ice until use. Viral challenge dose was quantitated using a Tissue Culture Infectious Dose 50% (TCID50) assay using the Reed and Muench method on Vero E6 cells in DMEM supplemented with 2% FBS and 100 IU/mL Penicillin G and 100 μg/mL Streptomycin. A challenge dose of 1.0×105 TCID50 per animal was targeted. Actual challenge dose averaged 5.8×105 TCID50 per animal. The viral challenge dose was delivered via intranasal installation under anesthesia (ketamine 80 mg per kg and xylazine 5 mg per kg) with a volume of 100 μL per nare (200 μL total per animal).

1.12 AUG-3387 Treatment

The AUG-3387 mAb was delivered by one of two routes for each animal, IT and IP injection. The IP injection was performed with a 16 mg/mL formulation in saline.

The intratracheal insufflation was performed with animals under anesthesia (4-5% isoflurane with oxygen) until a deep plane of anesthesia was reached. Dry powder for inhalation delivery was transferred to the ABSL-3 facility and each individual device quantitatively loaded for delivery. Doses were based on method development to quantify the amount of material that exited the devices assuming 100% presentation at the terminus of the canula and the animals average body weight during dosing.

1.13 Quantitative Assessment of Viral Burden

Quantitation of genomic viral RNA and subgenomic viral RNA, by RT-qPCR was used as markers for viral burden. Nasal swab and lung samples were assayed via RT-qPCR for both the N-gene (genomic) and the E-gene (subgenomic). For both methods, lung samples were weighed and homogenized using a Tissue Lyser (Qiagen) in 1 ml of TRI reagent. RNA was extracted using the Direct-Zol 96-RNA kit (Zymo Research) according to manufacturer's instructions. RNA was quantified using qRT-PCR TaqMan Fast Virus 1-step assay (Applied Biosystems). SARS-CoV-2 specific primers and probes from the 2019-nCoV RUO Assay kit (Integrated DNA Technologies) were used:

(L Primer: TTACAAACATTGGCCGCAAA (SEQ ID NO: 7);
R primer: GCGCGACATTCCGAAGAA (SEQ ID NO: 8);
probe: 6FAM-ACAATTTGCCCCCAGCGCTTCAG-BHQ-1 (SEQ
ID NO: 9)

Reactions were carried out on a BioRad CFX384 Touch instrument according to the manufacturer's specifications. A semi-logarithmic standard curve of synthesized SARS-CoV-2 N gene RNA (LBRI) was obtained by plotting the Ct values against the logarithm of cDNA concentration and used to calculate SARS-CoV-2 N gene in copies per gram of tissue or per nasal swab.

Copies of SARS-CoV-2 E gene were measured by qRT-PCR TaqMan Fast Virus 1-step assay (Thermo Fisher). SARS-CoV-2 specific primers and probes from the 2019-nCoV RUO Assay kit (Integrated DNA Technologies) were used:

L Primer:
(SEQ ID NO: 10);
CGATCTCTTGTAGATCTGTTCTC
R primer:
(SEQ ID NO: 11)
ATATTGCAGCAGTACGCACACA;
probe:
(SEQ ID NO: 12)
6FAM-ACACTAGCCATCCTTACTGCGCTTCG-BHQ-1.

Reactions were carried out on a BioRad CFX384 Touch instrument according to the manufacturer's specifications. A semi-logarithmic standard curve of synthesized SARS-CoV-2 E gene RNA (LBRI) was obtained by plotting the Ct values against the logarithm of cDNA concentration and used to calculate SARS-CoV-2 E gene in copies per gram of tissue or per nasal swab. Thermal cycling conditions involved 5 minutes at 50° C. for reverse transcription, followed by an initial denaturation step for 20 seconds at 95° C. and 40 cycles of 95° C. for 3 seconds and 60° C. for 30 seconds.

2. Results and Discussion

2.1. Antibody Isolation

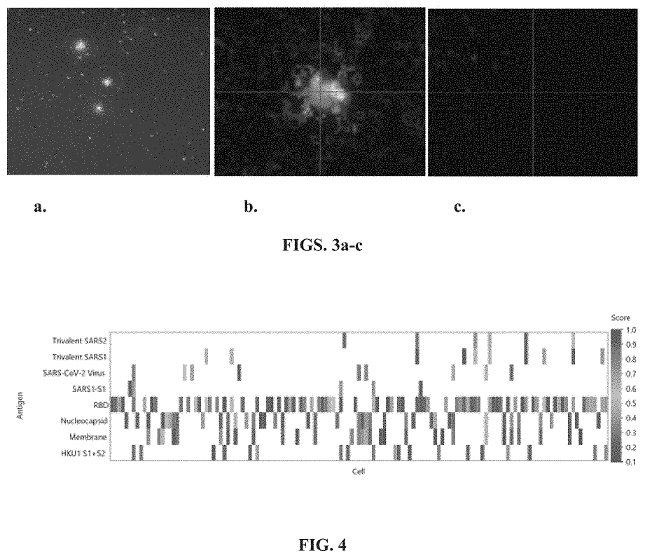

Approximately 800 single cells were isolated with SingleCyte and 200 with single cell flow sorting. From these cells, nearly 500 paired chain antibody constructs were designed and about 200 were expressed and assayed. many S1, S2, and RBD binders were recovered and ultimately chose AUG-3387 as the lead compound due to its breadth of binding activity, affinity to the Wuhan-1 strain, and strength in viral neutralization. FIGS. 3a-c illustrate (a) Raster of plate at 5× showing antigen specific B cells with signature reaction-diffusion pattern. (b,c) Before and after 20× false color images of automated capture of an S1-RBD specific plasma cell. Blue indicates antigen beads displaying 51 RBD protein, while green indicates beads displaying S2 protein. Magenta is a cell specific stain. FIG. 4 illustrates an exemplary output from a SingleCyte Screen. For each cell, the confidence of an antigen specific interaction is determined by the amount of secondary antibody signal that overlaps an antigen-specific bead image in the proximity of each cell. A score of 0 represents a 50% chance of antigen specificity, with 1.0 representing a 100% certainty.

2.2. Antibody Characterization

2.2.1 AUG-3387 Single Domain Affinity

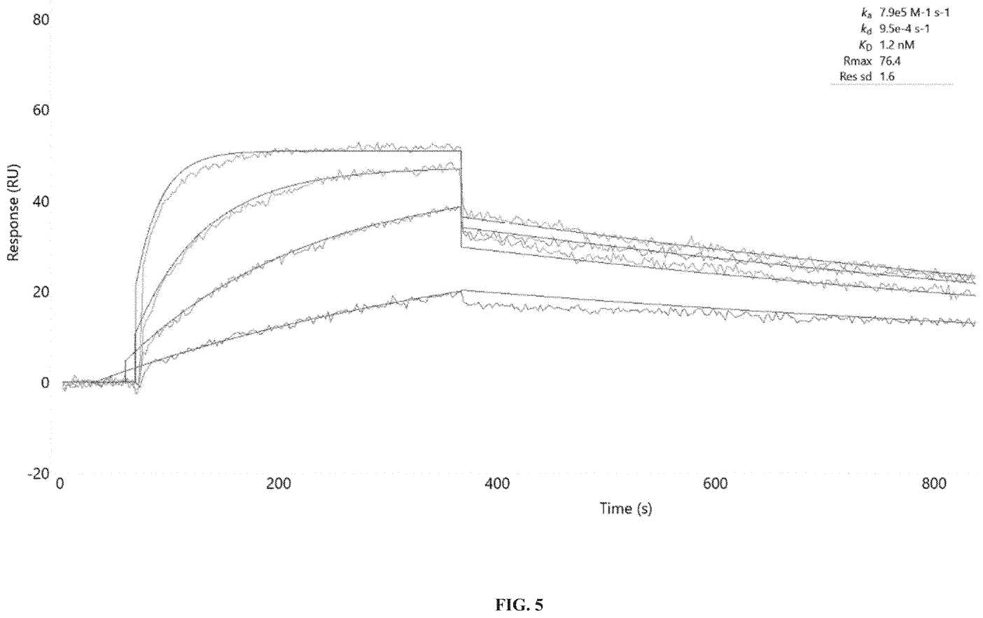

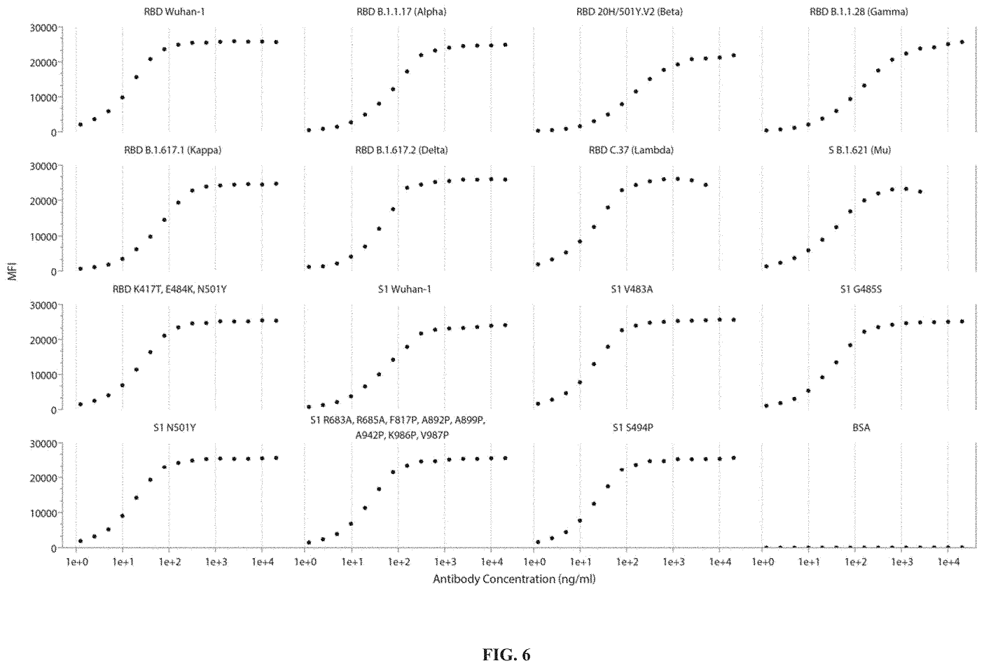

The Carterra LSA platform was employed to determine the single domain affinity of AUG-3387, expressed as an ScFv “AUG-3705”. See FIG. 5. Auto-fitting of curves was performed in Carterra Kinetics software, which returned a calculated affinity of 1.2 nM. See FIG. 5.

2.2.2 AUG-3387 Variant Binding Assays

To assess the susceptibility of AUG-3387 to mutational escape, AUG-3387 was profiled against the S1 and RBD portions of the original Wuhan-1 strain of SARS-CoV-2, RBD's corresponding to WHO designated dominant strains of concern, and S1 mutants known to affect the potency of currently approved therapeutic antibodies. AUG-3387 binds every S1 and RBD of SARS-CoV-2 tested with a binding EC50<200 ng/mL (FIG. 6), but only very weakly to SARS-CoV-1 (binding EC50>100 ug/ml, not shown). Although the problems with dose-response curves of solid phase immunoassays is well established, we gained confidence in this approach from the repeatability in performance across a range of antigens from the same supplier, variant susceptibility of our other antibodies, and confirmatory data (BLI and SPR). See FIG. 6.

2.2.3 AUG-3387 Neutralization of SARS-CoV-2 Wuhan-1

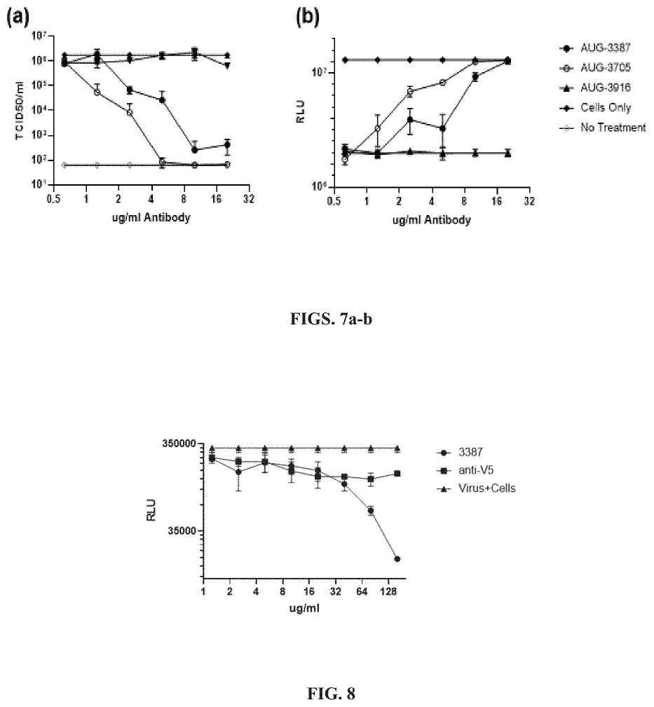

The ability of full length IgG1 formatted AUG-3387 and its ScFv formatted version, AUG-3705, was compared to neutralize live SARS-CoV-2 in a 24 hour TCID50 assay and a 96 hour infected cell viability assay. AUG-3705 demonstrated somewhat higher efficacy in these assays over AUG-3387, indicating the improved avidity of the dimeric IgG1 did not improve neutralization enough to compensate for the higher molarity of AUG-3705 at the same concentration. See FIG. 7.

2.2.3 AUG-3387 Neutralization of Delta Pseudovirus

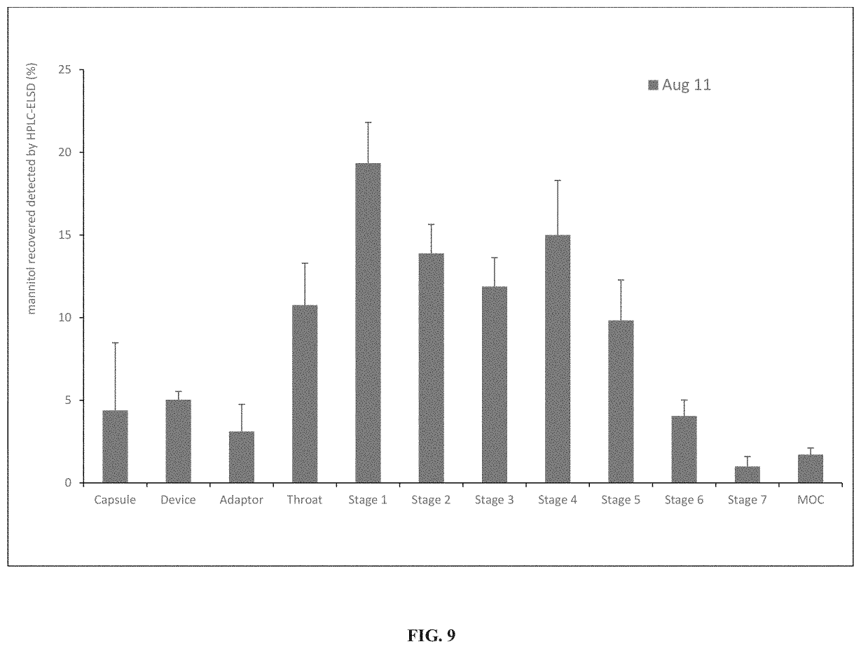

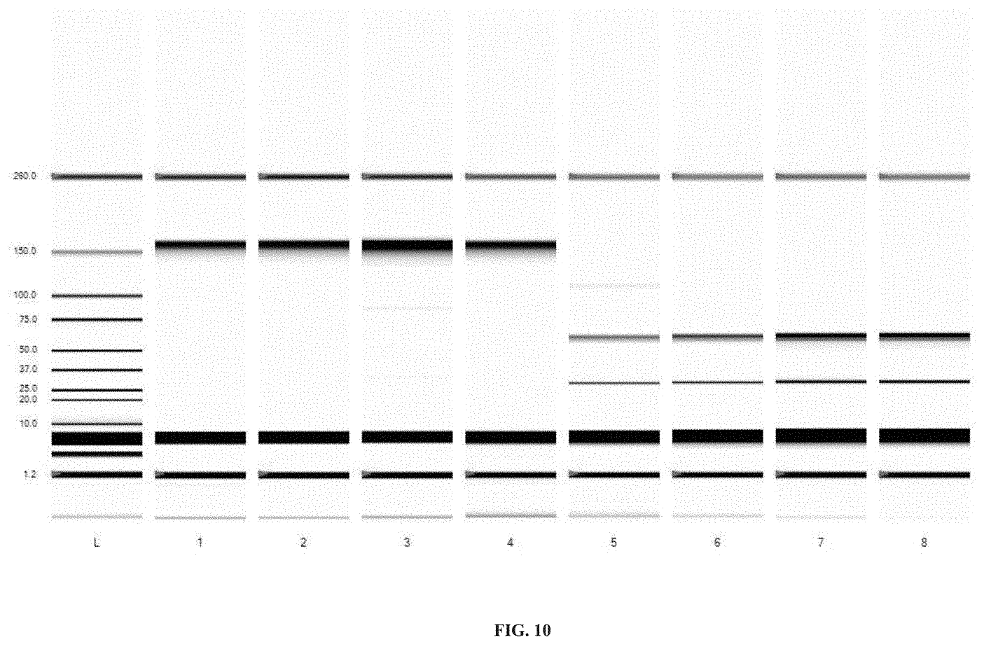

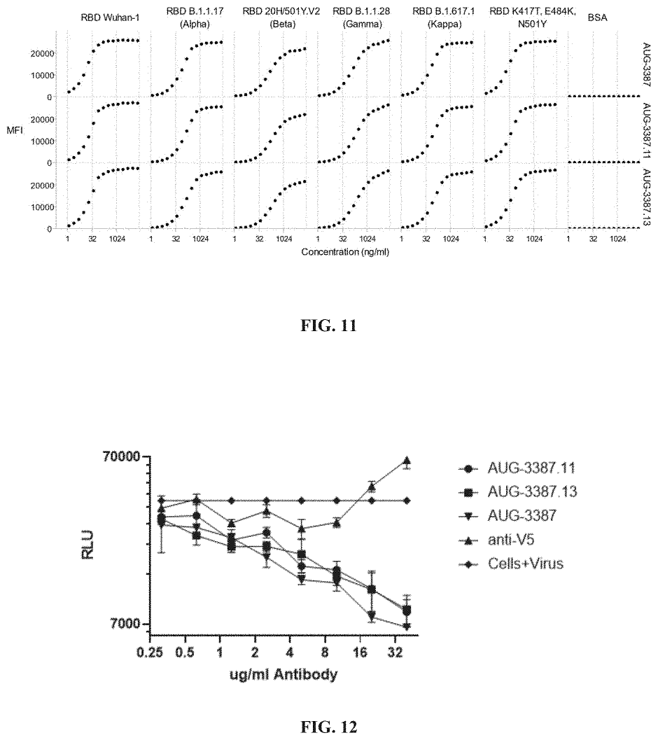

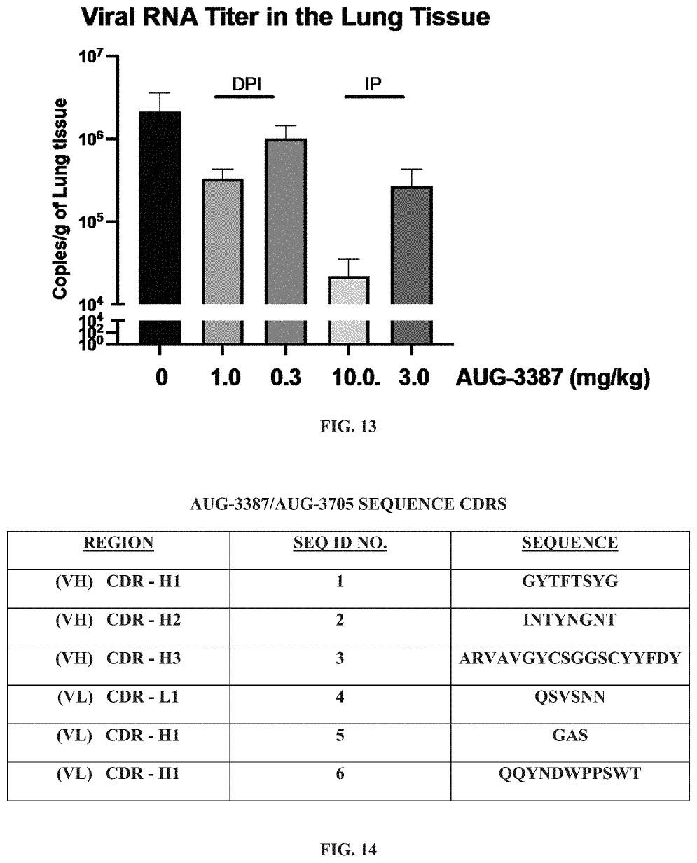

The ability of AUG-3387 was assessed to neutralize Delta pseudotyped virus (FIG. 8). AUG-3387 demonstrated the ability to neutralize Delta pseudovirus, albeit at higher IC50 (30-40 μg/mL) than for the Wuhan-1 strain (<1 μg/mL in TCID50 assay).

2.5. TFF Powder Optimization and Characterization

Thin-film freeze-dried AUG-3387 powders of various compositions that contained AUG-3387 at a range of mAb concentrations from 5-20% (w/w) and excipients. The powders were tested for the presences or absence of subvisible aggregates under a light microscope, for mAb aggregation or fragmentation using SDS-PAGE, and for their aerosol properties using the NGI. Ultimately, while mannitol/leucine (95%/5%) or trehalose/leucine (95%/5%) as excipients were evaluated as AUG-3387.11 and AUG-3387.13, respectively, the dry powder designated AUG-3387.11 prepared with mannitol/leucine (95%/5%) as the excipients with an AUG-3387 loading of 15% (w/w) was selected for additional studies because the dry powder showed excellent aerosol properties (FIG. 9). This powder delivered through a Plastiape RS00 Dry powder inhaler gave an MMAD value of 3.74±0.73 μm, GSD of 2.73±0.20, and an FPF (delivered) of 50.95±7.69%. Upon reconstitution of the powder, no significant subvisible aggregated particles in the solution were observed under a microscope.

Two formulations of AUG-3387 in TFF powders (AUG-3387.11 and AUG-3387.13) were assessed and compared them to the original AUG-3387 formulation in PBS. AUG-3387.11 and AUG-3387.13 performed virtually identically to their soluble counterpart in gel electrophoresis, multiplexed bead assays, and pseudoneutralization.

2.6 AUG-3387 Reduces SARS-CoV-2 Viral Load in Syrian Golden Hamsters

The efficacy of AUG-3387 for therapeutic reduction of viral load was assessed in vivo using the established hamster model with the mAb formulations being delivered starting 24 hours after intranasal SARS-CoV-2 inoculation. Hamsters were administered AUG-3387 at doses of 3 and 10 mg/kg or a vehicle control by intraperitoneal (IP) injection. Additional groups received three doses of the TFF dry powder formulation of AUG-3387 by intratracheal (IT) instillation of at doses of 0.3 and 1 mg/kg at 24, 48, and 72 hours after SARS-CoV-2 inoculation. All animals showed body weight loss. On Day 5, animals were harvested and lung tissues were assessed for viral replication by rt-qPCR for subgenomic (active) viral replication. Dose dependent viral load reductions were observed with both the IP and IT treated animals showing reduced viral load in the lung tissue.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A dry powder formulation for neutralizing SARS-CoV-2 and known variants thereof in a subject in need thereof comprising administering to the patient the dry powder composition comprising a human-derived monoclonal antibody AUG-3387.

2. The dry powder formulation of claim 1, wherein the dry powder formulation comprises less than 5% water.

3. The dry powder formulation of claim 1, wherein the dry powder formulation comprises less than 2% water.

4. The dry powder formulation of claim 1, wherein the dry powder formulation comprises particles having an average diameter of about 0.1 to about 100 mm.

5. The dry powder formulation of claim 1, wherein the dry powder formulation comprises particles having an average diameter of about 1 to about 50 mm.

6. The dry powder formulation of claim 1, wherein the dry powder formulation comprises particles having an average diameter of about 5 to about 15 mm.

7. The dry powder formulation of claim 1, wherein the dry powder formulation further comprises an excipient.

8. The dry powder formulation of claim 1, wherein the dry powder formulation is suitable for administration by inhalation.

9. The dry powder formulation of claim 8, wherein the inhalation is nasal administration.

10. The dry powder formulation of claim 8, wherein the inhalation is oral administration.

11. A method for neutralizing SARS-CoV-2 virus in a subject in need thereof, comprising administering to said subject a dry powder formulation comprising human-derived monoclonal antibody, AUG-3387.

12. The method of claim 11, wherein the dry powder is added to a pharmaceutically acceptable liquid to form a suspension.

13. The method of claim 11, wherein the suspension is administered to the subject by inhalation.

14. The method of claim 13, wherein the inhalation is nasal administration.

15. The method of claim 13, wherein the inhalation is oral administration.

16. The method of claim 11, wherein the pharmaceutically acceptable liquid is saline or sterile water.

17. The method of claim 11, wherein the dry powder formulation comprises less than 5% water.

18. The method of claim 11, wherein the dry powder formulation comprises particles having an average diameter of about 0.1 to about 100 mm.

19. The method of claim 11, wherein the dry powder formulation comprises particles having an average diameter of about 1 to about 50 mm.

20. The method of claim 11, wherein the dry powder formulation comprises particles having an average diameter of about 5 to about 15 mm.

21-43. (canceled)


SEQUENCE LISTING (12 sequences)

SEQ ID NO: 1 - protein, 8 aa, Homo sapiens
GYTFTSYG

SEQ ID NO: 2 - protein, 8 aa, Homo sapiens
INTYNGNT

SEQ ID NO: 3 - protein, 18 aa, Homo sapiens
ARVAVGYCSGGSCYYFDY

SEQ ID NO: 4 - protein, 6 aa, Homo sapiens
QSVSNN

SEQ ID NO: 5 - skipped

SEQ ID NO: 6 - protein, 11 aa, Homo sapiens
QQYNDWPPSWT

SEQ ID NO: 7 - DNA, 20 bp, synthetic construct
  source 1..20: mol_type = other DNA
ttacaaacattggccgcaaa

SEQ ID NO: 8 - DNA, 18 bp, synthetic construct
  source 1..18: mol_type = other DNA
gcgcgacattccgaagaa

SEQ ID NO: 9 - DNA, 23 bp, synthetic construct
  source 1..23: mol_type = other DNA
acaatttgcccccagcgcttcag

SEQ ID NO: 10 - DNA, 23 bp, synthetic construct
  source 1..23: mol_type = other DNA
cgatctcttgtagatctgttctc

SEQ ID NO: 11 - DNA, 22 bp, synthetic construct
  source 1..22: mol_type = other DNA
atattgcagcagtacgcacaca

SEQ ID NO: 12 - DNA, 26 bp, synthetic construct
  source 1..26: mol_type = other DNA
acactagccatccttactgcgcttcg